SEQUENCE LISTING (44 sequences)

SEQ ID NO: 1 - protein, 110 aa, synthetic construct
DIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTV

SEQ ID NO: 2 - protein, 118 aa, synthetic construct
QIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSS

SEQ ID NO: 3 - protein, 11 aa, synthetic construct
RASGNVHNYLA

SEQ ID NO: 4 - protein, 17 aa, synthetic construct
WINTETGEPTYTDDFKG

SEQ ID NO: 5 - DNA, 864 bp, synthetic construct
  source 1..864: mol_type = other DNA
tgccgccagaacacagctgggctagccgccaccatgatgtcctttgtctctctgctcctggttggcatcctattccatgccacccaggccgacatccagatgacccagtcccctgcctctctgtccgccagcgtgggcgagaccgtgacaatcacctgcagagcctctggcaacgtgcacaattacctggcttggtatcagcagaagcagggcaagtccccacagctgctggtgtacacagccaagaccctggctgacggcgtgcccagcaggttctctggctccggcagcggcacacagtatagcctgaagatcaactctctgcagcctgaggattttggcacctactattgccagcatttctggtctaatccatggacatttggcggcggcaccaagctggagatcaagaggacagtgggttcaacgtcaggctctggaaaacctggctcaggtgagggttccaccaaagggcagattcagctggtccagagcggtcccgagctgaagaagccaggcgagaccgtgaagatctcctgcaaggccagcggctacgctttcacagactattctatgaactgggtgaagcaggccccaggcaagggcctgaggtggatgggctggatcaataccgagacaggcgagcccacctacacagacgatttcaagggccggttcgctttttccctggagacctctgcctccacagcttttctgcagatcaacaatctgagaaacgaggacaccgccacatacttctgcgctaggcactacgattatggcggctttccttattggggccagggcaccctggtgacagtgtccagcgaatctaagtacggaccgccctgccccccttgccct

SEQ ID NO: 6 - DNA, 864 bp, synthetic construct
  source 1..864: mol_type = other DNA
tgccgccagaacacagctgggctagccgccaccatgatgtcctttgtctctctgctcctggttggcatcctattccatgccacccaggcccagattcagctggtccagagcggtcccgagctgaagaagccaggcgagaccgtgaagatctcctgcaaggccagcggctacgctttcacagactattctatgaactgggtgaagcaggccccaggcaagggcctgaggtggatgggctggatcaataccgagacaggcgagcccacctacacagacgatttcaagggccggttcgctttttccctggagacctctgcctccacagcttttctgcagatcaacaatctgagaaacgaggacaccgccacatacttctgcgctaggcactacgattatggcggctttccttattggggccagggcaccctggtgacagtgtccagcggttcaacgtcaggctctggaaaacctggctcaggtgagggttccaccaaaggggacatccagatgacccagtcccctgcctctctgtccgccagcgtgggcgagaccgtgacaatcacctgcagagcctctggcaacgtgcacaattacctggcttggtatcagcagaagcagggcaagtccccacagctgctggtgtacacagccaagaccctggctgacggcgtgcccagcaggttctctggctccggcagcggcacacagtatagcctgaagatcaactctctgcagcctgaggattttggcacctactattgccagcatttctggtctaatccatggacatttggcggcggcaccaagctggagatcaagaggacagtggaatctaagtacggaccgccctgccccccttgccct

SEQ ID NO: 7 - DNA, 8790 bp, synthetic construct
  source 1..8790: mol_type = other DNA
accgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgagtttcgttagcctcctcctcgttggcatactgtttcacgcaacgcaggcagacatccagatgactcagtctcctgcctccttgagtgcatccgtgggcgaaacagttaccatcacttgtagggccagcgggaacgtccataactacctggcctggtaccagcaaaagcaaggcaaaagcccccaacttctcgtttatacggcaaagaccctggcagatggagtgccctcacggttcagtggaagcggcagtggaacccaatatagcctgaaaatcaactctttgcaacctgaagattttgggacctattactgtcaacatttttggtctaatccttggacgtttggcgggggtaccaaactggaaattaagcggaccgtcggcagtacaagtggctcagggaagcccggaagtggcgaagggagtactaaagggcagatccagcttgttcaatctggacctgagttgaaaaaaccgggggagacagtgaaaatatcttgtaaggcatctgggtatgcgttcacggactacagtatgaactgggtgaagcaggctcctgggaagggccttcggtggatgggatggatcaatacggaaaccggggaaccaacatatacagatgatttcaagggccgcttcgcatttagtctggaaacatctgcgtctaccgcattcctgcaaattaacaatttgcggaacgaggataccgctacgtacttctgtgcgcgacactatgattatggagggttcccgtactggggacagggtactctggtcacggtttccagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggata

SEQ ID NO: 8 - DNA, 9111 bp, synthetic construct
  source 1..9111: mol_type = other DNA
agcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgagtttcgttagcctcctcctcgttggcatactgtttcacgcaacgcaggcagacatccagatgactcagtctcctgcctccttgagtgcatccgtgggcgaaacagttaccatcacttgtagggccagcgggaacgtccataactacctggcctggtaccagcaaaagcaaggcaaaagcccccaacttctcgtttatacggcaaagaccctggcagatggagtgccctcacggttcagtggaagcggcagtggaacccaatatagcctgaaaatcaactctttgcaacctgaagattttgggacctattactgtcaacatttttggtctaatccttggacgtttggcgggggtaccaaactggaaattaagcggaccgtcggcagtacaagtggctcagggaagcccggaagtggcgaagggagtactaaagggcagatccagcttgttcaatctggacctgagttgaaaaaaccgggggagacagtgaaaatatcttgtaaggcatctgggtatgcgttcacggactacagtatgaactgggtgaagcaggctcctgggaagggccttcggtggatgggatggatcaatacggaaaccggggaaccaacatatacagatgatttcaagggccgcttcgcatttagtctggaaacatctgcgtctaccgcattcctgcaaattaacaatttgcggaacgaggataccgctacgtacttctgtgcgcgacactatgattatggagggttcccgtactggggacagggtactctggtcacggtttccagcgaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtg

SEQ ID NO: 9 - DNA, 9441 bp, synthetic construct
  source 1..9441: mol_type = other DNA
attaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgagtttcgttagcctcctcctcgttggcatactgtttcacgcaacgcaggcagacatccagatgactcagtctcctgcctccttgagtgcatccgtgggcgaaacagttaccatcacttgtagggccagcgggaacgtccataactacctggcctggtaccagcaaaagcaaggcaaaagcccccaacttctcgtttatacggcaaagaccctggcagatggagtgccctcacggttcagtggaagcggcagtggaacccaatatagcctgaaaatcaactctttgcaacctgaagattttgggacctattactgtcaacatttttggtctaatccttggacgtttggcgggggtaccaaactggaaattaagcggaccgtcggcagtacaagtggctcagggaagcccggaagtggcgaagggagtactaaagggcagatccagcttgttcaatctggacctgagttgaaaaaaccgggggagacagtgaaaatatcttgtaaggcatctgggtatgcgttcacggactacagtatgaactgggtgaagcaggctcctgggaagggccttcggtggatgggatggatcaatacggaaaccggggaaccaacatatacagatgatttcaagggccgcttcgcatttagtctggaaacatctgcgtctaccgcattcctgcaaattaacaatttgcggaacgaggataccgctacgtacttctgtgcgcgacactatgattatggagggttcccgtactggggacagggtactctggtcacggtttccagcgaatctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattc

SEQ ID NO: 10 - DNA, 8790 bp, synthetic construct
  source 1..8790: mol_type = other DNA
attaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgtccttcgtttcactcctgctggtggggatactcttccacgcgacccaagcccagatccagcttgtacaatccggcccagaattgaaaaaacccggtgagacggttaagatttcatgcaaagccagtgggtatgctttcaccgactatagtatgaattgggtaaagcaggcacctgggaaagggctcaggtggatggggtggattaacacggagactggtgaacccacctacaccgacgattttaagggccgctttgcattctctcttgagacatccgcctccacggcatttttgcaaataaataatctgcggaatgaggatacggcgacctacttctgcgcaaggcattacgattacggcggcttcccatactggggccaggggactctcgtcactgtcagctccggtagtactagcgggagcgggaaaccgggtagtggtgaaggctctacaaaaggggatatacaaatgacccaatctccggcgtccctctctgcaagcgtgggcgagactgttacaattacgtgcagggcgagcgggaacgtacataactacttggcgtggtaccaacaaaaacaagggaaaagccctcagctgctcgtatatacagcaaaaactttggccgatggagtcccgagcaggttcagtggaagtggaagtgggactcaatactctttgaagataaacagtcttcaaccagaagatttcgggacgtactactgtcaacacttttggagtaatccctggacgtttggaggcggcactaagctggagatcaagcgcacggtggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattc

SEQ ID NO: 11 - DNA, 9111 bp, synthetic construct
  source 1..9111: mol_type = other DNA
agcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgtccttcgtttcactcctgctggtggggatactcttccacgcgacccaagcccagatccagcttgtacaatccggcccagaattgaaaaaacccggtgagacggttaagatttcatgcaaagccagtgggtatgctttcaccgactatagtatgaattgggtaaagcaggcacctgggaaagggctcaggtggatggggtggattaacacggagactggtgaacccacctacaccgacgattttaagggccgctttgcattctctcttgagacatccgcctccacggcatttttgcaaataaataatctgcggaatgaggatacggcgacctacttctgcgcaaggcattacgattacggcggcttcccatactggggccaggggactctcgtcactgtcagctccggtagtactagcgggagcgggaaaccgggtagtggtgaaggctctacaaaaggggatatacaaatgacccaatctccggcgtccctctctgcaagcgtgggcgagactgttacaattacgtgcagggcgagcgggaacgtacataactacttggcgtggtaccaacaaaaacaagggaaaagccctcagctgctcgtatatacagcaaaaactttggccgatggagtcccgagcaggttcagtggaagtggaagtgggactcaatactctttgaagataaacagtcttcaaccagaagatttcgggacgtactactgtcaacacttttggagtaatccctggacgtttggaggcggcactaagctggagatcaagcgcacggtggaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtg

SEQ ID NO: 12 - DNA, 9441 bp, synthetic construct
  source 1..9441: mol_type = other DNA
attaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgggctagccgccaccatgatgtccttcgtttcactcctgctggtggggatactcttccacgcgacccaagcccagatccagcttgtacaatccggcccagaattgaaaaaacccggtgagacggttaagatttcatgcaaagccagtgggtatgctttcaccgactatagtatgaattgggtaaagcaggcacctgggaaagggctcaggtggatggggtggattaacacggagactggtgaacccacctacaccgacgattttaagggccgctttgcattctctcttgagacatccgcctccacggcatttttgcaaataaataatctgcggaatgaggatacggcgacctacttctgcgcaaggcattacgattacggcggcttcccatactggggccaggggactctcgtcactgtcagctccggtagtactagcgggagcgggaaaccgggtagtggtgaaggctctacaaaaggggatatacaaatgacccaatctccggcgtccctctctgcaagcgtgggcgagactgttacaattacgtgcagggcgagcgggaacgtacataactacttggcgtggtaccaacaaaaacaagggaaaagccctcagctgctcgtatatacagcaaaaactttggccgatggagtcccgagcaggttcagtggaagtggaagtgggactcaatactctttgaagataaacagtcttcaaccagaagatttcgggacgtactactgtcaacacttttggagtaatccctggacgtttggaggcggcactaagctggagatcaagcgcacggtggaatctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtaccgagctcggatccactagtccagtgtggtggaattctgcagatatccagcacagtggcggccactcaagtctggagggcacgttaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattc

SEQ ID NO: 13 - protein, 265 aa, Synthetic construct
MMSFVSLLLVGILFHATQADIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVGSTSGSGKPGSGEGSTKGQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSS

SEQ ID NO: 14 - protein, 265 aa, Homo sapiens
MMSFVSLLLVGILFHATQAQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSGSTSGSGKPGSGEGSTKGDIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTV

SEQ ID NO: 15 - protein, 12 aa, Homo sapiens
ESKYGPPCPPCP

SEQ ID NO: 16 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 229 aa, Homo sapiens
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 28 aa, Homo sapiens
MFWVLVVVGGVLACYSLLVTVAFIIFWV

SEQ ID NO: 19 - protein, 111 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 20 - protein, 42 aa, Homo sapiens
KRGRKKLLYIFKQPFMRPVQTTQEEDGCSCRFPEEEEGGCEL

SEQ ID NO: 21 - protein, 840 aa, Synthetic construct
MMSFVSLLLVGILFHATQADIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVGSTSGSGKPGSGEGSTKGQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 22 - protein, 947 aa, Synthetic construct
MMSFVSLLLVGILFHATQADIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVGSTSGSGKPGSGEGSTKGQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 23 - protein, 1057 aa, Synthetic construct
MMSFVSLLLVGILFHATQADIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVGSTSGSGKPGSGEGSTKGQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - protein, 840 aa, Synthetic construct
MMSFVSLLLVGILFHATQAQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSGSTSGSGKPGSGEGSTKGDIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 25 - protein, 947 aa, Synthetic construct
MMSFVSLLLVGILFHATQAQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSGSTSGSGKPGSGEGSTKGDIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 26 - protein, 1057 aa, Synthetic construct
MMSFVSLLLVGILFHATQAQIQLVQSGPELKKPGETVKISCKASGYAFTDYSMNWVKQAPGKGLRWMGWINTETGEPTYTDDFKGRFAFSLETSASTAFLQINNLRNEDTATYFCARHYDYGGFPYWGQGTLVTVSSGSTSGSGKPGSGEGSTKGDIQMTQSPASLSASVGETVTITCRASGNVHNYLAWYQQKQGKSPQLLVYTAKTLADGVPSRFSGSGSGTQYSLKINSLQPEDFGTYYCQHFWSNPWTFGGGTKLEIKRTVESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 27 - protein, 19 aa, Synthetic construct
MMSFVSLLLVGILFHATQA

SEQ ID NO: 28 - protein, 265 aa, synthetic construct; identical to SEQ ID NO: 13

SEQ ID NO: 29 - protein, 265 aa, Synthetic construct; identical to SEQ ID NO: 14

SEQ ID NO: 30 - protein, 15 aa, Homo sapiens
EPKSCDKTHTCPPCP

SEQ ID NO: 31 - protein, 12 aa, Homo sapiens
ERKCCVECPPCP

SEQ ID NO: 32 - protein, 61 aa, Homo sapiens
LKTPLGDTTHTCPRCPEPKSCDTPPPCPRCPEPKSCDTPPPCPRCPEPKSCDTPPPCPRCP

SEQ ID NO: 33 - protein, 12 aa, Homo sapiens
ESKYGPPCPSCP

SEQ ID NO: 34 - protein, 21 aa, Homo sapiens
PVPSTPPTPSPSTPPTPSPSC

SEQ ID NO: 35 - protein, 58 aa, Homo sapiens
ESPKAQASSVPTAQPQAEGSLAKATTAPATTRNTGRGGEEKKKEKEKEEQEERETKTP

SEQ ID NO: 36 - protein, 112 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 37 - protein, 99 aa, Homo sapiens
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 38 - protein, 15 aa, Homo sapiens
KHLCPSPLFPGPSKP

SEQ ID NO: 39 - protein, 48 aa, Homo sapiens
AKPTTTPAPRPPTPAPTIASQPLSLRPEACRPAAGGAVHTRGLDFACD

SEQ ID NO: 40 - protein, 45 aa, Homo sapiens
TTTPAPRPPTPAPTIASQPLSLRPEACRPAAGGAVHTRGLDFACD

SEQ ID NO: 41 - protein, 38 aa, Homo sapiens
SVVDFLPTTAQPTKKSTLKKRVCRLPRPETQKGPLCSP

SEQ ID NO: 42 - protein, 36 aa, Homo sapiens
VDFLPTTAQPTKKSTLKKRVCRLPRPETQKGPLCSP

SEQ ID NO: 43 - protein, 36 aa, Homo sapiens
APPRASALPAPPTGSALPDPQTASALPDPPAASALP

SEQ ID NO: 44 - protein, 23 aa, Homo sapiens
DSGQVLLESNIKVLPTWSTPVQP

(19) United States
(12) Patent Application Publication
(10) Pub. No.: US 2026/0207665 A1
(43) Pub. Date: Jul. 23, 2026

(54) METHODS FOR DIAGNOSING AND TREATING OVARIAN CANCER

(71) Applicant: Carina Biotech Limited, Mawson Lakes (AU)

(72) Inventors: Martin Klaus Oehler, St Georges (AU); Carmela D'Antuono, Parkside (AU); Lizamarie Shenali Bernadine Goonetilleke, Somerton Park (AU); Kanchana Veronika Bandara, Beulah Park (AU); Simon Charles Barry, Longwood (AU); Timothy John Sadlon, Maylands (AU)

(21) Appl. No.: 19/137,022

(22) PCT Filed: Dec. 7, 2023
(86) PCT No.: PCT/AU2023/051266

§ 371 Date: Jun. 9, 2025

(30) Foreign Application Priority Data
Dec. 9, 2022 (AU) 2022903762

(51) Int. Cl. A61K 35/17; A61K 40/15; A61K 40/31; A61K 40/42; A61P 35/00; C07K 16/30; C12Q 1/6886; G01N 33/575; G01N 33/57545

(52) U.S. Cl. CPC A61K 35/17; A61K 40/15; A61K 40/31; A61K 40/4261; A61P 35/00; C07K 16/303; C12Q 1/6886; G01N 33/57545; G01N 33/5759; A61K 2239/13; A61K 2239/21; A61K 2239/59; C07K 2317/55; C07K 2317/622; G01N 2333/705

(57) ABSTRACT

Provided are methods for the prognosis and/or diagnosis of cancer, particularly ovarian cancer. Also provided are methods for the treatment of ovarian cancer as well as Chimeric Antigen Receptors (CARs) and CAR-expressing cells for the treatment of cancer, particularly ovarian cancer.

This application claims priority from Australian provisional patent application 2022903762 filed on 9 Dec. 2022, the entire disclosure of which is herein incorporated by way of this reference.

### TECHNICAL FIELD

The present invention relates to methods for diagnosing, treating, and determining the prognosis of ovarian cancer. Particularly the present invention relates to identification of glypican-1 expression on ovarian cancers, such as high-grade serous ovarian cancer, and uses of immunotherapies in the treatment of glypican-1 positive cancer.

### BACKGROUND OF INVENTION

The following discussion of the background to the invention is intended to facilitate an understanding of the invention. However, it should be appreciated that the discussion is not an acknowledgement or admission that any of the material referred to was published, known or part of the common general knowledge as at the priority date of any one of the claims of this specification.

Ovarian cancer is the most lethal gynaecological malignancy. High-grade serous ovarian cancer (HGSOC) makes up nearly 70% of ovarian cancer and 90% of patients present with advanced stage disease. Current treatment for HGSOC consists of de-bulking surgery followed by combined platinum and taxane-based chemotherapy. Although the initial response to first-line treatment is high, over 75% of patients eventually relapse and acquire chemotherapy resistance, which is the primary cause of ovarian cancer death and a major limitation to the successful treatment of ovarian cancer.

Currently there is a lack of options for the treatment of ovarian cancer, in particular relapsed ovarian cancer. Further, there is a lack of biomarkers to allow diagnosis of, or indication of the likely prognosis of, ovarian cancer. Therefore, there is a need to provide alternatives to current treatments (such as surgery and chemotherapy) for the treatment of ovarian cancer. There is also a need for improved diagnostics or prognostics for ovarian cancer.

### SUMMARY OF INVENTION

The present invention is predicated on the surprising finding that the Heparan sulfate proteoglycan, Glypican-1 (GPC1), is associated with ovarian cancer, including high-grade serous ovarian cancer.

Accordingly, in one aspect there is provided a Chimeric Antigen Receptor (CAR) comprising an antigen-recognition domain, a transmembrane domain, and a signalling domain, wherein the antigen-recognition domain recognises Glypican-1 (GPC1). The CAR, when expressed by an appropriate cell, can be used to target Glypican-1 expressing cancer cells, particularly ovarian cancer cells.

The antigen recognition domain can be any appropriate binding molecule which recognises Glypican-1, however in a preferred embodiment the antigen-recognition domain comprises a binding-portion of an antibody that recognises Glypican-1. In some embodiments, this portion of the antibody is selected from the group consisting of a fragment-antigen binding (Fab), a variable heavy chain of an antibody or a variable light chain of an antibody.

The antigen-recognition domain can also be a fusion protein, such as a single chain variable fraction (scFv) which has sequence identity to the variable heavy and light chains of an antibody that binds to Glypican-1.

Preferably, the CAR comprises a linker between the antigen-recognition domain and the transmembrane domain. In some embodiments, this linker comprises the IgG4 hinge region and/or the IgG4 CH3 region and/or the IgG4 CH2 region (which maybe included the mutations L235D or N297Q)

The present disclosure also provides a cell comprising the CAR of the invention. Chimeric antigen receptor constructs can be transduced into a variety of cell types, with particularly envisaged embodiments being an immune cell, such as lymphocytes, a CD3+ lymphocyte, a CD8+ lymphocyte (such as a CD8+ T cell), a CD4+ lymphocyte (such as a CD4+ T cell), a Natural killer (NK) cell or an NKT cell.

Also provided is use of the CAR, or a cell expressing the CAR, for the treatment or prevention of ovarian cancer in a subject, preferably wherein the ovarian cancer has elevated expression of Glypican-1.

Also provided is a method of diagnosing or assessing the prognosis of a subject having ovarian cancer, the method comprising determining the level of Glypican-1 in ovarian cells, or suspected cancer cells, from the subject, wherein an elevated level of Glypican-1 indicates the presence of ovarian cancer and/or indicates a worse prognosis for the subject. In some embodiments, a worse prognosis indicates a lower overall survival or a lower progression-free survival time.

In some embodiments, elevated gene expression of Glypican-1 indicates a lower overall survival. In some embodiments, elevated gene expression of Glypican-1 and/or elevated expression of Glypican-1 protein indicates a lower progression-free survival time.

In some embodiments, the ovarian cancer is relapsed ovarian cancer.

In some embodiments, the ovarian cancer is high-grade serous ovarian cancer.

In some embodiments, the method of diagnosis or assessment of prognosis is performed on a subject that has previously been treated for ovarian cancer with one or more of: chemotherapy, surgical resection or de-bulking, or radiotherapy.

In some embodiments, the ovarian cancer is relapsed ovarian cancer and the elevated level of Glypican-1 is compared to cancer tissue prior to relapse.

In some embodiments, the elevated level of Glypican-1 is compared to non-cancerous ovarian tissue.

In some embodiments, determining the level of Glypican-1 comprises quantifying Glypican-1 protein expression and/or mRNA expression. Glypican-1 protein expression can be the surface expression of the Glypican-1 protein and/or the intracellular expression of the Glypican-1 protein, and/or secreted levels of Glypican-1.

In some embodiments, the protein expression is determined by an agent that preferentially, or selectively, binds to Glypican 1. Such agents include antibodies or binding fragments of antibodies.

In some embodiments, the agent that binds to Glypican 1 is a fusion protein such as a single chain variable fragment which comprises the sequence of the variable light chain and variable heavy chain of an antibody.

In some embodiments, the antibody is MIL-38.

The invention also provides a method of treating a subject having ovarian cancer, or preventing ovarian cancer in a subject, the method comprising killing cells expressing Glypican-1. In some embodiments of this method, the cells expressing Glypican-1 are determined to express an elevated level of Glypican-1 protein.

In some embodiments, the elevated level of Glypican-1 protein expression includes elevated surface expression of the Glypican-1.

In some embodiments of the method of treatment, the ovarian cancer is relapsed ovarian cancer.

In some embodiments of the method of treatment, the ovarian cancer is high-grade serous ovarian cancer (HSOC).

In some embodiments of the method of treatment, the subject has previously been treated for ovarian cancer with one or more of chemotherapy, surgical resection or de-bulking, or radiotherapy.

In some embodiments of the method of treatment, the ovarian cancer is relapsed ovarian cancer and the elevated level of Glypican-1 is compared to cancer tissue prior to relapse and/or compared to non-cancerous ovarian tissue.

In some embodiments of the method of treatment, the cells are killed by administering to the subject, or inducing in a subject, an agent that preferentially, or selectively binds to Glypican-1 expressed by cells. Such agents may, in some embodiments, be an antibody or a binding fragment of an antibody. In some embodiments, the agent that binds to Glypican 1 may be a fusion protein such as a single chain variable fragment comprising the sequence of the variable light chain and variable heavy chain of an antibody. In some embodiments, the antibody is a humanised antibody. One particularly envisaged antibody is the antibody is MIL-38.

In some embodiment of the method of treatment, the agent that binds to Glypican-1 is a chimeric antigen receptor (CAR) expressing cell. In some embodiments, the CAR is the CAR disclosed herein.

In some embodiments of treatment, the method of diagnosing or assessing the prognosis (as disclosed herein) is performed, prior to killing the cells expressing Glypican-1.

Also provided is use of a CAR or agent directed against Glypican-1 in the manufacture of a medicament for the prevention or treatment of ovarian cancer in a subject, preferably wherein the ovarian cancer has elevated expression of Glypican-1. Also provided is a method for the prevention or treatment of ovarian cancer in a subject, preferably wherein the ovarian cancer has elevated expression of Glypican-1.

### BRIEF DESCRIPTION OF DRAWINGS

For a further understanding of the aspects and advantages of the present invention, reference should be made to the following detailed description, taken in conjunction with the accompanying drawings.

FIG. 1A to 1F: GPC1 expression is increased in HGSOC tissues compared to non-cancer tissues.

(A) GPC1 mRNA expression data obtained from the GENT2 database including ovarian surface epithelium (OSE) (n=66), fallopian tube (FT) (n=40) and high-grade serous ovarian cancer HGSOC (n=807). Higher GPC1 expression was observed in HGSOC compared to FT (****P<0.0001, Kruskal Wallis with Dunn's multiple comparison test). (B) GPC 1 H-Index scores of OSE, benign serous cystadenoma and (HGSOC) tissues assessed by GPC1 immunohistochemistry (IHC) staining and measured via Qupath, Data presented as mean±SEM. (*P<0.05, One way ANOVA with Tukey's multiple comparison test). Representative images of GPC1 protein expression by IHC in (C) OSE, (D) FT, (E) benign serous cystadenoma and (F) HGSOC (scale bar=50 μm) All images are of the same magnification.

FIG. 2A to 2F: High GPC1 expression is associated with poor prognosis.

Kaplan Meier survival analysis of the relationship between GPC1 mRNA expression and progression-free survival (PFS) and overall survival (OS) in patients with HGSOC. (A) PFS HR=1.3, 95% CI, 1.1-1.53, p=0.0015, n=1029) and (B) OS (HR=1.3, (95% CI, 1.15-1.58, p=0.00026, n=1144). Data was assessed using the online Kaplan Meier tool. Representative images of (C) HGSOC with low GPC1 protein expression and (D) HGSOC with high GPC1 expression by IHC (scaled bar=50 μm), all images are of the same magnification. Kaplan Meier survival analysis shows the relationship between GPC1 protein expression and HGSOC PFS and OS. Maximum H-index scores were used as cut-off points to separate samples into groups with high (H index>70) or low (H-index≤70) GPC1 protein expression. (E) PFS, log rank test, P=0.031, (n=96) (F) OS, log rank test, p=0.536, (n=100).

FIG. 3A to 3E: GPC1 is highly expressed in relapse tissues compared to tissues at diagnosis in matching HGSOC patients.

Representative images of GPC1 protein expression in HGSOC tissues at (A-B) diagnosis and at (C-D) relapse by IHC (scale bar 50 μm). All images are of the same magnification. (E) GPC1 staining in HGSOC tissues at diagnosis (n=4) and relapse (n=4) were measured using QuPath (*P<0.05, paired T-test).

FIG. 4A to 4F: GPC1 expression in ovarian cancer cells.

GPC1 mRNA expression quantitation in (A) ovarian cancer cell lines and (B) primary HGSOC cells from (n=4) independent experiments performed in triplicate by qRT-PCR. Data was analyzed using the 2^{-Δct} method and normalized to the housekeeper gene β-actin. GPC1 protein levels were assessed by western blotting GPC1 (65 kDa) and β-actin (48 kDa), 20 μg of protein from each ovarian cancer cell line (C) and primary ovarian cancer cells (D) was loaded and electrophoresed onto a 4-20% TGX gel. Quantification of western blots for (E) ovarian cancer cell lines (n=9), and (F) primary cells (n=7), from 2 to 3 independent gels. (Data presented as mean±SD) normalized to β-actin).

FIG. 5A to 5F: Effect of GPC1 CAR-T cell treatment on ovarian cancer survival in a 2D monolayer assay.

Ovarian cancer cells were plated at 10,000 cells/well in 96 well plates and treated with media alone, untransduced (UT) CD3 T-cells (blue) or GPC1 CAR-T cells (pink) (ratio 2:1, 5:1 & 10:1, E:T ratio) for 48 hours. Cell survival was calculated using an MTT assay. (A) OVCAR3 (B) COV362, (C) OV90, (D). SKOV-3, (E) Patient 1 and (F) Patient 3. Data presented as mean±SD (*P<0.05, unpaired t-test from 3 independent experiments). GPC1 CAR-T cells displayed potent killing effects at all concentrations in SKOV3 and COV362 cell lines compared to UT T-CD3 cells. OV90 and OVCAR3 cells treated with 5:1 and 2:1 GPC1 CAR T cells resulted in a significant decrease in cell survival compared to UT CD3 T-cells. Both Patient 1 and 3 cells exhibited a significant decrease in cell survival at 10:1 but did not display any significant decrease in cell survival at 5:1 and patient 3 alone exhibited a decrease in cell survival at 2:1.

FIG. 6A to 6C: Effect of GPC1 CAR-T cells on ovarian cancer 3D spheroid cultures.

Representative image of (A) COV362, (B) SKOV3, (C) OVCAR-3 at 48 hours post-treatment with media alone, untransduced (UT) CD3 T-cells (5:1) or GPC1 CAR-T cells (5:1). Spheroid images were collected from 3 individual experiments in duplicate, and measurements were taken using ImageJ software from 5 randomly selected areas within each well. Data was expressed as a percentage of control of the spheroid area. A significant decrease in spheroid size was observed between the UT CD3 T-cell group and the GPC1 CAR-T cell group for all cell lines. Statistical significance was assessed by One-way ANOVA with Tukey's multiple comparison test. *P<0.05, **P<0.01, *** P<0.001, scale bar represents 1000 μm. All images are of the same magnification.

FIGS. 7A and 7B: Effect of GPC1 CAR-T cells on primary ovarian cancer cells 3D spheroid cultures.

Representative images of (A) patient 1 and (B) patient 3 spheroids cultured at 48 hours post-treatment with media alone, untransduced (UT) CD3 T-cells (5:1) or GPC1 CAR-T cells (5:1). Spheroid images were collected from 3 individual experiments in duplicate, and measurements were taken using ImageJ from 5 randomly selected areas within each well. Data were expressed as a percentage of control of the spheroid area. A significant decrease in spheroid size was observed between the control group and the GPC1 CAR-T cell treatment group for both primary cells. Statistical significance was assessed by One-way ANOVA with Tukey's multiple comparison test. *P<0.05, **P<0.01. Scale bar represents 1000 μm, all images are of the same magnification.

FIG. 8A to 8G: Effects of GPC1 CAR-T cells in patient-derived explant assays

GPC1 CART cell treatment induces apoptosis in patient derived explants with high GPC1 expression. A-F Cleaved caspase 3 quantitation, data is expressed as % of positively stained cell/mm2, the bar graphs show the mean values+/−SD. Cleaved caspase 3 expression is significantly increased in the CAR-T treatment group compared to the untransduced group, unpaired t-test for patients 1~4 (A-D) but not patients 5 and 6 (E-F). G. Representative images of GPC1 expression by immunohistochemistry in an explant tissue that didn't not respond and an explant tissue that responded to treatment GPC1 CAR-T cells. Scale bar=50 μm, all images are of the same magnification. GPC1 expression measured by H score using QuPath was significantly increased in responders compared to non-responders.

FIG. 9: Development of GPC1 CD3 CAR-T cells

Flow chart of the established protocols for preparation of CAR-T cells in Professor Simon Barry's Laboratory.

FIG. 10: Flow Cytometric analysis (FACs) of batch 98 cells

EGFR expression in CD3 untransduced (UT) T-cells and GPC1 CD3 CART cells

FIG. 11: Flow cytometric analysis (FACs) of batch 98 cells-Cell Maturation panel

CD45RA and CD62L markers in CD4 T cell population and CD8 T cell population. Q9: T_{EMRA} (effector memory cells re-expressing CD45RA, CD45RA+CD62L−), Q10: Naïve phenotype (CD45RA+CD62L+), Q11: central memory (CD45RA−CD62L+), Q12: effector memory (CD45RA−CD62L−)

FIGS. 12A and 12B: Flow cytometric analysis (FACs) of batch 2 cells. Exhaustion panel.

(A) Programmed cell death-1 (PD1), LAG3 and TIM expression in CD4 T cell population. (B) Programmed cell death-1 (PD1), LAG3 and TIM expression in CD8 T cell population

FIGS. 13A and 13B: GPC1 expression in controls

Representative images of GPC1 protein expression in mouse kidney as used for the IHC analyses. (A) positive control, and (B) negative control.

FIGS. 14A and 14B: Relationship of GPC1 with progression-free survival and overall survival using quartile measurements for H-index.

Quartile color legend: 1 (blue), 2 (red), 3 (green), 4 (orange). A. Relationship of GPC1 tumor measurement with progression-free survival (n=93). P=0.363, H-index quartile 1 (15/21), H-index quartile 2 (19/25), H-index quartile 3 (19/23), H-index quartile 4 (17/24). B. Relationship of GPC1 tumor measurements overall survival (n=100), p=0.973, H-index quartile 1 (17/25), H-index quartile 2 (16/25), H-index quartile 3 (17/25), H-index quartile 4 (16/25).

FIGS. 15A to 15F: Construction of CNA500200, CNA510200, CNA 500300, CNA510300, CNA500400 and CNA510400.

Six different CAR constructs were generated. These consisted of two different scFv fusion proteins providing the binding domain, and three different linker domains. The two scFvs comprised: (i) the MIL-38 leader sequence (1) linked to the variable light (VL) chain of the MIL-38 antibody (2) which was fused via the Whitlow linker (3) to the variable heavy (VH) chain (4) of the MIL-38 antibody; and (ii) the MIL-38 leader sequence (1) linked to the variable heavy (VH) chain (4) of the MIL-38 antibody which was fused via the Whitlow linker (3) to the variable light (VL) chain (2) of the MIL-38 antibody. The three linked domains comprise: (i) IgG4 hinge (5), (ii) IgG4 hinge+IgG4 CH3 (11) and (iii) IgG4 hinge+IgG4 CH2 L235D and N297Q mutations +IgG4 CH3 (12). The CARs further included a CD28 transmembrane domain (6), a costimulation domain having a portion of 4-1BB (7), an activation domain having a portion of CD3zeta (8), a T2A self-cleavage site (9) and a truncated EGFR (10).

### DETAILED DESCRIPTION

The present invention is predicated, in part, on the recognition by the inventors that Glypican-1 (GPC1) is expressed by ovarian cancer cells. Accordingly, GPC1 expression can provide information on the presence of disease in an individual. Additionally, the inventors have demonstrated that GPC1 can also act as a prognostic marker for patients suffering from ovarian cancer with elevated expression indicating worse outcomes in patients.

Moreover, the present inventors have demonstrated that CPG1 can be targeted to kill cancer cells, such as ovarian cancer cells, and therefore can provide a target for cancer treatment.

### Glypican-1

Glypican proteins are within the Heparan sulfate proteoglycans (HSPGs) families and are numbered 1 (GPC-1) through to 6 (GPC-6).

Glypican-1 (GPC1) is a glycosylphosphatidylinositol-anchored heparan sulfate proteoglycan. Its cDNA sequence is set forth in NCBI Reference Sequence: NM_002081.3, and its protein sequence is set forth in NCBI Reference Sequence: NP_002072.2. It consists of a 558-amino-acid core protein with three predicted heparan sulfate chains, which are attached at S486, S488, and S490, and has both a membrane-anchored form (by GPI at S530) and a secreted soluble form.

During embryonic development, GPC1 is mainly expressed in the neural and skeletal systems and at low levels in adult tissues, such as the heart and testes and it participates in organ development by modulating extracellular growth signals and morphogen gradient formation.

### Treatment of Cancer

As indicated above, and as exemplified herein, GPC1 represents a target for cancer cell therapy.

In some aspects, the present invention provides a method of treating a subject having cancer, the method comprising killing cells expressing Glypican-1.

Also provided is a method of treating or preventing cancer in a subject, the method comprising administering to the subject, or inducing in a subject, an agent that targets Glypican-1.

The inventors have demonstrated that glypican-1 is associated with ovarian cancer. Accordingly, in some embodiments of the methods, the cancer is ovarian cancer. In some embodiments of the methods, the ovarian cancer is relapsed ovarian cancer.

Most epithelial ovarian/fallopian tube cancers are the serous type, and they are graded as low-grade serous carcinoma (LGSC or LSOC) or high-grade serous carcinoma (HGSC or HSOC). These tumours have different genetic alterations and biology.

The Inventors have shown that Glypican-1 is particularly associated with high-grade serous ovarian cancer. Accordingly, in some embodiments of the methods of treatment or prevention, the ovarian cancer is an epithelial cell ovarian cancer, particularly a serous ovarian cancer, most particularly a high-grade serous ovarian cancer. In some embodiments, the ovarian cancer is germ cell ovarian cancer. In some embodiments, the ovarian cancer is a stromal cell ovarian cancer.

In some embodiments, the ovarian cancer is relapsed ovarian cancer.

Agents suitable for targeting or killing cells expressing Glypican-1 include, but are not limited to: antibodies, and binding fragments thereof, antibody-drug-conjugates (ADCs), radionuclide labelled antigen binding molecules, fusion proteins, chimeric antigen receptor (CAR) expressing cells, bispecific binding molecules including bispecific T cell engagers and bispecific antibodies, and vaccines designed to initiate an immune response against GPC1.

Accordingly, in some embodiments of the methods of treatment or prevention, the subject is administered, or the cells are exposed to, an agent that preferentially, or selectively, binds to Glypican 1. Such agents may, in some embodiments, be an antibody or a binding fragment of an antibody.

Antibody binding fragments can be derived from an antibody or may be recombinantly generated with sequences identical to the CDRs of an antibody or antibody fragment. Indeed, these CDRs may be from an affinity matured antibody and therefore may not be identical to an in vivo derived antibody.

Antibodies are comprised of four chains (two heavy and two light chains) and can be separated into the Fc (fraction crystallisable) and the Fab (fraction antigen binding) domains. The Fc portion of the antibody interacts with Fc receptors and the complement system. Consequently, the Fc portion is important for the immune function of the antibody. However, the Fab portion contains the binding regions of the antibody and is critical for the specificity of an antibody for the desired epitope.

Accordingly, in some embodiments, antibody fragment is a Fab fragment of an antibody. Fab fragments can be individual Fab fragments (i.e., the antibody fragment is generated in the absence of linking disulphide bridges) or an F(ab′) 2 fragment which comprises the two Fab fragments of an antibody linked via disulphide bridges. These fragments are typically generated by fragmenting an antibody using digestion enzymes, such as pepsin. Methods are known in the art for preparing such Fabs (see for example see Sjögren, J. et al., Methods Mol Biol. 2017; 1535: pp. 319-329).

Antibodies consist of six CDRs in total with the VH and VL chains comprising three CDRs each (within a framework consisting of 4 framework regions). Individual VH and VL chains (each only comprising three CDRs) have been shown to bind specifically with high affinity. Typically, individual binding regions are known as single antibody domains (sdAbs). Alternatively, the VH and VL chains can be linked via a linker to form a fusion protein known as a single-chain variable fragment (scFv—also known as a diabody). Unlike Fabs, scFvs are not fragmented from an antibody, but rather are typically recombinantly formed based on the CDR and framework regions of an antibody. Further, sdAbs and scFvs can also be recombinantly produced and form the binding component of a larger fusion protein which may also include additional portions. Consequently, in some embodiments, the agent is, or includes, an scFv or a sdAb including CDRs from antibodies that bind to GPC1. The scFv may include multiple VH and VL chains linked together to form a multivalent scFv, such as a di-scFv or a tri-scFv.

In some embodiments the antibody that binds to GPC1 is MIL-38 (Miltuximab™).

In some embodiments, the agent that binds to Glypican 1 may be a fusion protein such as a single chain variable fragment comprising the sequence of the variable light chain and variable heavy chain of an antibody, such as the antibody MIL-38. In some embodiments, the agent that bind to Glypican-1 includes a VH or a VL chain of an antibody that binds to Glypican-1, such as MIL-38.

Antibodies to specific analytes may be obtained commercially or generated by methods known in the art. For example, antibodies to specific analytes may be prepared using methods generally disclosed by Howard and Kaser (Making and Using Antibodies: a Practical Handbook, CRC Press, 2007).

In some embodiments, the variable heavy region has the amino acid sequence of SEQ ID No. 2, or a variant thereof having sequence identity to this sequence. In some embodiments, the variable light region has the amino acid sequence of SEQ ID No. 1, or a variant having sequence identity to this sequence. In some embodiments, the variants of the variable heavy or variable light chains are at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 98.2%, at least 98.4%, at least 98.6%, at least 98.8%, at least 99%, at least 99.1%, at least 99.2%, at least 99.3%, at least 99.4%, at least 99.5%, at least 99.6%, at least 99.7%, at least 99.8%, at least 99.9% identical to the variable heavy and/or variable light chains of SEQ ID No. 2 and/or SEQ ID No. 1.

In some embodiments, the variable heavy region includes a heavy chain CDR1 having the amino acid sequence DYSMN or having the amino acid sequence DYSMN with up to 1, 2 or 3, amino acid modifications, a heavy chain CDR2 having an amino acid sequence as set forth in SEQ ID No. 4, or having an amino acid sequence as set forth in SEQ ID No. 4 with up to 1, 2 or 3 amino acid modifications, and a heavy chain CDR3 having the amino acid sequence HYDYGGFPY, or having the amino acid sequence HYDYGGFPY with up to 1, 2 or 3 amino acid modifications.

In some embodiments, the variable light chain includes a light chain CDR1 having an amino acid sequence set forth in SEQ ID No. 3 or having an amino acid sequence as set forth in SEQ ID No. 3 with up to 1, 2 or 3 amino acid modifications, a light chain CDR2 having the amino acid sequence TAKTLAD or having the amino acid sequence TAKTLAD with up to 1, 2 or 3 amino acid modifications, and a light chain CDR3 having the amino acid sequence QHFWSNPWT or having the amino acid sequence QHFWSNPWT with up to 1, 2 or 3 amino acid modifications.

In some embodiments, the antibody or antigen binding fragment includes heavy chain CDR1, CDR2 and CDR3 having the amino acid sequence of DYSMN, SEQ ID No: 4 and HYDYGGFPY, with up to 1, 2 or 3 amino acid modifications.

In some embodiments, the antibody or antigen binding fragment includes light chain CDR1, CDR2 and CDR3 having an amino acid sequence as set forth in SEQ ID No. 3, TAKTLAD and QHFWSNPWT, with up to 1, 2 or 3 amino acid modifications.

The specificity, avidity and affinity of antibodies generated within subjects can be modified by way of in vitro processes such as affinity maturation (see for example; Fujino Y. et al. Biochem Biophys Res Comm., 2012; 428 (3): 395-400; Li, B. et al. MAbs. 2014; 6 (2): pp. 437-45 and Ho M and Pastan I, “In vitro Antibody Affinity Maturation Targeting Germline Hotspots”, Method Mol Biol., 2009; 525:293-xiv). These techniques include (but are not limited to) site-directed mutagenesis and PCR-driven mutagenisis, phage library development and affinity screening. For example, mutation adjacent to hotspot locations defined by A/G-G-C/T-A/T (RGYW) and AG-C/T (AGY) sequences (with reference to the coding immunoglobulin DNA) are likely to modify the affinity of produced antibodies. Alternatively, processes such as in vitro scanning saturation mutagenesis (Chen, G et al. Protein Eng Des Sel., 1999; (12) 4:346-356) can be used to replace each and every modification within a CDR region with each other possible mutation. Each variant then assessed for antigen affinity and specificity. As such, in vivo derived antibodies, or binding fragments thereof, can be further modified to produce distinct, yet lineally related, antibodies. Consequently, the term “antibody” (and fragments thereof) encompasses in vivo derived antibodies and in vitro derived molecules that have undergone processes of mutation to modify the CDR binding sites, such that they have unique sequences when compared to the antibodies generated in vivo. Further, binding portions of antibodies, in particular the CDRs, can be affinity matured and mutated using techniques known in the art.

The term “antibody” also includes non-conventional antibodies generated from species such as camelids, shark and jawfish. As such, the term antibody includes heavy-chain antibodies including camelid antibodies, IgNARs and variable lymphocyte receptors (VLRs). Further, these can be fragmented into their biding portions (such as VNARs—single binding portion of IgNARs) or integrated recombinantly into a fusion protein. Methods are known in the art for generating and adapting such non-conventional antibodies, for example see Nuttall, S., Methods Mol. Biol, 2012; 911: pp. 27-36 and Vincke C. et al., Methods Mol. Biol. 2012; 907: pp. 145-76.

Antibodies can be generated which bind to GPC1. Further, such antibodies can be affinity matured to optimise abiding affinity and avidity. Therefore, in some embodiments the binding domain includes a sequence identical to the binding region(s) of an antibody that binds to GPC1, or includes a sequence corresponding to an affinity matured form of the binding region that binds to GPC1. While affinity matured binding regions can significantly vary from the original antibody binding regions, in preferred forms the affinity mature form of the binding region has at least 80%, 85%, 90%, 95% 97%, 98% or 99% sequence identity to an antibody that binds to GPC1.

In some embodiments, the agent that binds to Glypican-1 is an Antibody-Drug Conjugate (ADC).

Antibody-drug-conjugate use an immunoconjugate in which a cytotoxic agent is chemically or enzymatically linked to an antibody that selectively binds to an internalizing tumor-associated antigen, thereby selectively delivering a cytotoxic agent to specific cells. Most ADCs comprise and IgG1 antibody conjugated to a microtubule inhibitor such as maytansine or auristatin.

Other known conjugates for use in ADCs include: Monomethyl auristatin E (MMAE—Used in Brentuximab Vedotin (Adcetris™) and Enfortumab Vedotin (Padcev™); Monomethyl auristatin F (MMAF)—Used in Belantamab mafodotin (Blenrep™); Calicheamicin—Used in Gemtuzumab Ozogamicin (Mylotarg™); Maytansinoid DM1-Used in Trastuzumab Emtansine (Kadcyla™); Maytansinoid DM4—Used in Mirvetuximab Soravtansine; Pyrrolobenzodiazepine (PBD) dimers-Used in Rova-T; camptothecin analogues—used in Sacituzumab Govitecan (Trodelvy™); Duocarmycin analogs-used in Trastuzumab duocarmazine; Camptothecin derivative SN-38—used in Sacituzumab Govitecan (Trodelvy™); Irinotecan metabolite SN-38—Used in Trastuzumab Deruxtecan (Enhertu™); and Topoisomerase I inhibitor-Used in Trastuzumab Deruxtecan (Enhertu™). Any of the listed conjugates can be used in the present invention. Further conjugates and information on the preparation of such ADCs are provided in Riccardi F, et al. (2023), A comprehensive overview on antibody-drug conjugates: from the conceptualization to cancer therapy. Front Pharmacol, 14:1274088, the contents of which are incorporated herein.

Antibody-drug-conjugates for Glypican-1 are known in the art. These include those disclosed in: Matsuzaki S, et al. (2017). Anti-glypican-1 antibody-drug conjugate exhibits potent preclinical antitumor activity against glypican-1 positive uterine cervical cancer. Int J Cancer, 1; 142 (5), 1056-1066; Yokota K, et al. (2021). Anti-Glypican-1 Antibody-drug Conjugate as Potential Therapy Against Tumor Cells and Tumor Vasculature for Glypican-1-Positive Cholangiocarcinoma. Mol Cancer Ther, 20 (9), 1713-1722; Munekage E, et al. (2021). A glypican-1-targeted antibody-drug conjugate exhibits potent tumor growth inhibition in glypican-1-positive pancreatic cancer and esophageal squamous cell carcinoma. Neoplasia, 23 (9), 939-950; and Tsujii S et al. (2021). Glypican-1 Is a Novel Target for Stroma and Tumor Cell Dual-Targeting Antibody-Drug Conjugates in Pancreatic Cancer. Mol Cancer Ther, 20 (12), 2495-2505 (the contents of the above listed references are included herein).

The methods of treatment or prevention provided herein may be used in patients diagnosed with cancer—particularly ovarian cancer. In some embodiments, the methods of treatment or prevention are performed after analysing the expression of Glypican-1 in the subject or, specifically, on the cancer cells of the subject. In some embodiments of the method of prevention or treatment, the cells expressing Glypican-1 are determined to express an elevated level of Glypican-1, in particular Glypican-1 protein, or Glypican-1 mRNA. What constitutes elevated levels of Glypican-I are known in the art and are defined herein. Methods for assessing the level of protein and mRNA are known in the art and are provided herein.

In some embodiments, the elevated level of Glypican-1 protein expression includes elevated surface expression of the Glypican-1 protein and/or elevated intracellular expression of Glypican-1 protein.

In some embodiments of the methods of treatment, the ovarian cancer is relapsed ovarian cancer and the elevated level of Glypican-1 is compared to cancer tissue prior to relapse and/or compared to non-cancerous ovarian tissue.

In some embodiments of the methods of prevention or treatment, a method of diagnosis or prognosis as described herein is performed prior to treatment, or after treatment.

The methods of treatment and prevention of the present invention can be performed alone or in combination with other treatments for cancer. Such treatments include, but are not limited to chemotherapy, surgical resection or de-bulking, or radiotherapy.

In some embodiments, the present methods of treatment or prevention is performed as an adjuvant therapy in combination with another treatment such as immunotherapy. In some embodiments, the agent that binds to Glypican 1 is administered together with an immune checkpoint inhibitor such as PD1 inhibitors, PD-L1 inhibitors, CTLA4 inhibitors, LAG3 inhibitors, TIM3 inhibitors or TIGIT inhibitors, including antibodies or binding agents that bind to these targets.

In some embodiments of the method of treatment, the agent that binds to Glypican-1 is a chimeric antigen receptor (CAR) (expressed on a cell). In some embodiments, the CAR is the anti-GPC1 CAR as disclosed herein.

### Chimeric Antigen Receptor

Chimeric antigen receptors (CARs) are artificially constructed proteins that upon expression on the surface of a cell can induce an antigen-specific cellular response. In their most basic form CARs include at least three domains. The first domain being an extracellular antigen-recognition domain that specifically recognises an antigen, or more specifically an epitope portion, or portions, of an antigen. The second domain being an intracellular signalling domain that is capable of inducing, or participating in the induction, of an intracellular signalling pathway. And the third domain being a transmembrane domain that traverses the plasma membrane and bridges the extracellular antigen-recognition domain and the intracellular signalling domain.

The combination of the first two domains determines the antigen specificity of the CAR and the ability of the CAR to induce a desired cellular response, the latter of which is also dependent on the host cell of the CAR. For example, the activation of a CAR expressed in a T-helper cell and having a signalling domain comprising a CD3 activation domain may-once activated by its cognate antigen-induce the CD4+T-helper cell to secrete a range of cytokines. In a further example, the same CAR when expressed in a CD8+ cytotoxic T cell—once activated by a cell expressing the cognate antigen—may induce the release of cytotoxins that ultimately lead to the induction of apoptosis of the antigen-expressing cell.

The third domain (the transmembrane domain) may comprise a portion of, or may be associated with, the signalling domain of the CAR. The transmembrane domain is typically one or more hydrophobic helices, which spans the lipid bilayer of a cell and embeds the CAR within the cell membrane. The transmembrane domain of the CAR can be one determinant in the expression pattern of the CAR when associated with a cell. For example, using a transmembrane domain associated with a CD3 co-receptor can permit expression of the CAR in naïve T cells, amongst others, whilst use of a transmembrane domain from a CD4 co-receptor may direct expression of a CAR in T-helper cells. Use of the CD8 co receptor transmembrane domain may direct expression in cytotoxic T lymphocytes (CTLs), while the CD28 transmembrane domain may permit expression in both CTLs and T helper cells and can assist in stabilising the CAR.

A further component, or portion, of a chimeric antigen receptor may be a linker domain. The linker domain spans from the extracellular side of the transmembrane domain to the antigen-recognition domain, thereby linking the antigen-recognition domain to the transmembrane domain. Typically, in the art, the linker domain is considered as an optional domain, as some CARs function without a linker domain.

Accordingly, in one aspect, the present invention provides a Chimeric Antigen Receptor (CAR) comprising an antigen-recognition domain, a transmembrane domain, and a signalling domain, wherein the antigen-recognition domain recognises Glypican-1 (GPC1).

As used throughout the specification the term “recognises” (in relation to Glypican-1) refers to the ability of the binding domain to associate with a desired epitope of GPC1 or to any portion of the GPC1 molecule. Preferably this recognition is selective, in that the binding domain binds exclusively, or predominantly, to GPC1. In some embodiments, the binding domain may directly bind to GPC1, or an epitope thereof. In some embodiments, the binding domain may indirectly bind to GPC1, or an epitope thereof, for example by way of an intermediate or bispecific molecule (for example a 5th generation CAR). In some embodiments, the antigen-recognition domain may bind to a processed form of GPC1. As used in this context, the term “processed form” relates to forms of GPC1 which have been truncated or digested, typically, as a result of intracellular processing including forms and epitopes of GPC1 which are presented on major histocompatibility complexes (e.g., human leukocyte antigens).

The CAR binding domain can be any suitable domain that can recognise GPC1, or an epitope thereof. As used throughout the specification the term “binding domain” refers to the portion of the CAR that provides the specificity of the CAR for GPC1. The binding domain, in the context of the present invention, only comprises a portion of the extracellular region (or ectodomain) of the CAR.

The binding domain of the CAR can comprise a range of binding molecules. These include antibodies (including non-conventional antibodies, such as heavy chain antibodies), antibody binding fragments (as described herein, including scFv, Fabs, sdAbs), and protein binding scaffolds. In some embodiments, the binding domain includes the variable heavy chain of an antibody that binds to GPC1 and/or the binding domain includes the variable light chain of an antibody that binds to GPC1-including the antibodies and binding fragments disclosed herein. In some embodiments, the binding domain includes an Fab. The antigen-recognition domain can also be a fusion protein, such as a single chain variable fraction (scFv) which has sequence identity to an antibody that binds to Glypican-1. In some embodiments the antigen binding domain comprises SEQ ID NO: 28 or 29 (with or without the MIL-38 leader of SEQ ID NO: 27), or a functional variant thereof having at least 50%, 60%, 70%, 80%, 90%, 95%, 98%, 99% or 99.5% sequence identity.

For the avoidance of doubt, the binding domain of the CAR can comprise any antibody or antibody fragment sequences (including CDRs) disclosed herein in relation to anti-GPC1 antibodies (such as those described as possible “agents”), including any possible modification disclosed herein.

Antibodies that can bind to GPC1 are discussed herein and include MIL-38.

### Linker Domain

The linker domain connects the transmembrane domain and antigen recognition domain of the CAR. CAR T cells have been formed that function without the inclusion of a linker domain, and therefore, in this context, a linker domain is not considered to be generally essential to the function of all CARS.

Without wanting to be bound by theory, a linker domain may provide an appropriate molecular length to the ectodomain (extracellular domain) of the CAR to allow recognition of the epitope by the antigen recognition domain, while forming the correct immunological synaptic distance between the effector cell expressing the CAR, and the target cell. Further, the linker domain may provide the appropriate flexibility for the antigen recognition domain to be orientated in the correct manner to recognise its epitope.

Therefore, in some embodiments, the extracellular domain includes a linker domain which links the binding domain to the transmembrane domain. In some embodiments, the linked domain is at least 12 amino acids in length. In some embodiments, the linked domain is at least about 12 amino acids in length. In some embodiments, the linked domain is greater than 12 amino acids in length. In some embodiments, the linked domain is at least 119 amino acids in length. In some embodiments, the linked domain is at least about 119 amino acids in length. In some embodiments, the linked domain is greater than 119 amino acids in length. In some embodiments, the linked domain is at least 229 amino acids in length. In some embodiments, the linked domain is at least about 229 amino acids in length. In some embodiments, the linked domain is greater than 229 amino acids in length.

In some embodiments, the linked domain is up to 119 amino acids in length. In some embodiments, the linked domain is up to about 119 amino acids in length. In some embodiments, the linked domain is up to 229 amino acids in length. In some embodiments, the linked domain is up to about 229 amino acids in length.

The selection of a suitable linker domain may be based on (i) reducing binding affinity to Fc Receptors (such as the Fcγ and FcRn receptor), which minimizes ‘off-target’ activation of CAR expressing cells and (ii) optimizing the efficacy of the CAR construct by enhancing the flexibility of the antigen binding region, reducing spatial constraints for formation of an immune synapse (e.g., reducing steric hindrance and optimising synaptic distance).

In some embodiments, the linker domain includes a sequence identical to a hinge region from an immunoglobulin, or a hinge or extracellular region from a membrane bound molecule involved in the formation of a T cell synapse. For example, the linker domain may comprise a region having an amino acid sequence homologous to a hinge region from CD4, CD8, CD3, CD7 or CD28.

In some embodiments, the linker domain includes a sequence identical to a portion of an immunoglobulin. In some embodiments, the portion is one or more of a hinge region (for example the IgG4 hinge region or a modified version thereof), a constant heavy (CH) 1 region, a CH2 region, a CH3 region or a CH4 region. In some embodiments, the portion is a CH2 region, a CH3 region or a hinge region of an immunoglobulin or has at least 50%, 60%, 70%, 80%, 90%, 95%, 98% or 99% sequence identity with said CH regions. In some embodiments, the portion is a CH2 region or a CH3 region and a hinge region of an immunoglobulin. In some embodiments, the immunoglobulin is selected from the IgG subtype.

In some embodiments, the linker domain includes a sequence having similarity to a portion of one or more of IgG1, IgG2, IgG3 or IgG4 Fc regions, for example the IgG1 hinge region and the CH2 or CH3 regions of IgG4 or functional variants thereof having at least 50%, 60%, 70%, 80%, 90%, 95%, 98%, 99% or 99.5% sequence identity.

In some embodiments, the linker domain includes a sequence identical to an immunoglobulin CH3 domain, an immunoglobulin CH2 domain or both a CH2 and CH3 domain. In some embodiments, the linker domain includes a sequence identical to an immunoglobulin hinge region and one or more of a CH3 domain or a CH2 domain. In some embodiments the CH2 and/or CH3 regions are from the IgG4 subclass of IgG antibodies.

In some embodiments, the linker domain includes all, or part of, an immunoglobulin hinge region. As would be understood in the art, the specific region that forms the hinge region of an immunoglobulin varies for different isotypes. For example, IgA, IgD and IgG isotype immunoglobulins have a hinge region between the CH1 and CH2 regions, while the function of the hinge region is provided by the CH2 region in IgE and IgM isotype immunoglobulins.

In some embodiments, this linker comprises the IgG4 hinge region and/or the IgG4 CH3 region and/or the IgG4 CH2 region (which maybe included the mutations L235D, N297Q).

A non-exhaustive list of sequences which may be incorporated into the linker domain is provided in Table 1, below. In some embodiments, the linker domain of the present invention may include any one or more of the components provided in Table 1. In some embodiments, the linker domain may consist of any one or more of the linkers provided in Table 1. Further, the linker domain may be an artificially synthesized sequence such poly-Glycine sequences or repeats of GGGGS (Gly4Ser) sequences (for example a (Gly4Ser) 3).

TABLE 1
Possible linker domain components

| Name | length | Sequence | SEQ ID |
|---|---|---|---|
| IgG1 | 15 | EPKSCDKTHTCPPCP | 30 |
| hinge | | | |
| IgG2 | 12 | ERKCCVECPPCP | 31 |
| hinge | | | |
| IgG3 | 62 | LKTPLGDTTHTCPRCPEPKSCDTPPPCPRCPEPK | 32 |
| hinge | | SCDTPPPCPRCPEPKSCDTPPPCPRCP | |
| IgG4 | 12 | ESKYGPPCPSCP | 33 |
| hinge | | | |
| IgG4 | 12 | ESKYGPPCPPCP | 15 |
| hinge | | | |
| (mutated) | | | |
| IgA1 | 21 | PVPSTPPTPSPSTPPTPSPSC | 34 |
| hinge | | | |
| IgD | 58 | ESPKAQASSVPTAQPQAEGSLAKATTAPATTRNT | 35 |
| hinge | | GRGGEEKKKEKEKEEQEERETKTP | |
| IgM | 112 | IAELPPKVSVFVPPRDGFFGNPRKSKLICQATGFS | 36 |
| common | | PRQIQVSWLREGKQVGSGVTTDQVQAEAKESGP | |
| heavy | | TTYKVTSTLTIKESDWLGQSMFTCRVDHRGLTFQ | |
| (CH) 2 | | QNASSMCVPD | |
| IgE CH2 | 99 | PTVKILQSSCDGGGHFPPTIQLLCLVSGYTPGTINI | 37 |
| | | TWLEDGQVMDVDLSTASTTQEGELASTQSELTL | |
| | | SQKHWLSDRTYTCQVTYQGHTFEDSTKKCA | |
| CD28 | 15 | KHLCPSPLFPGPSKP | 38 |
| Hinge | | | |
| CD8a | 48 | AKPTTTPAPRPPTPAPTIASQPLSLRPEACRPAA | 39 |
| Hinge (1) | | GGAVHTRGLDFACD | |
| CD8a | 45 | TTTPAPRPPTPAPTIASQPLSLRPEACRPAAGGA | 40 |
| Hinge (2) | | VHTRGLDFACD | |
| CD8b | 38 | SVVDFLPTTAQPTKKSTLKKRVCRLPRP | 41 |
| Hinge (1) | | ETQKGPLCSP | |
| CD8b | 36 | VDFLPTTAQPTKKSTLKKRVCRLPRP | 42 |
| Hinge (2) | | ETQKGPLCSP | |
| CD7 | 36 | APPRASALPAPPTGSALPDPQTASALPDPPAASA | 43 |
| | | LP | |
| CD4 | 23 | DSGQVLLESNIKVLPTWSTPVQP | 44 |
| Hinge | | | |
| PolyG/S | 5+ | (GGGGS)ₙ | |

The hinge region, CH2 and CH3 region of immunoglobulins, in particular IgG isotype antibodies, may be bound by Fc receptors such as Fc gamma receptors and Fc neonatal receptors. Binding of the linker domain of a chimeric antigen receptor can reduce the efficacy of the receptor and can lead to off target killing. Therefore, in some embodiments, the linker domain is designed such that it has a reduced, or no, capacity to bind with an Fc receptor. In some embodiments, the linker domain is identical to an immunoglobulin with a reduced capacity to bind with an Fc receptor compared to other immunoglobulin isotypes. In some embodiments, the linker domain of the chimeric antigen receptor does not comprise an amino acid sequence that substantially binds with an Fc receptor.

The ability for Fc receptors to bind with different IgG isotypes is presented in Table 2 below.

TABLE 2
Fc Receptor binding to IgG subtypes

| | IgG1 | IgG2 | IgG3 | IgG4 |
|---|---|---|---|---|
| FcγRIa (CD64) | +++ | − | ++++ | ++ |
| FcγRIIaa (CD32) | +++ | ++ | ++++ | ++ |
| FcγRIIba (CD32) | + | − | ++ | + |
| FcγRIIIba (CD16a) | +++ | − | ++++ | − |
| FcRn | +++ | +++ | +++ | +++ |

In some embodiments, where the linker domain includes a portion identical to the Fc region of an immunoglobulin, the portion maybe modified to reduce binding to the Fc receptor. Methods are known in the art to modify a protein to reduce binding by Fc Receptors. Fc gamma receptor primarily binds to the lower hinge region and the n-terminal of the CH2 region of immunoglobulin regions, while the neonatal Fc receptor primarily binds to amino acids at the C-terminus of the CH2 region and the N-terminus of the CH3 region. A guide to the binding of Fc receptors to IgG antibodies can be found in Chapter 7 of “Antibody Fc: Linking Adaptive and Innate Immunity” Ackerman and Nimmerjahn, Elsevier Science & Technology 2014. Therefore, modifications in these areas may alter the binding of Fc receptors to linker domains having homology with the Fc portion of immunoglobulins. A non-exhaustive exemplary list of mutations to Human IgG1, which have been shown to reduce Fc-gamma receptor and FcRn binding include: E116P, L117V, L118A, G119 deleted, P121A, S122A, I136A, S137A, R138A, T139A, E141A, D148A, S150A, S150A, E152A, D153A, E155A, N159A, D163A, H168A, N169A, K171A, K173A, R175A, E176A, Q178A, Y179F, N180A, S181A, R184A, V188A, T190A, L192A, Q194A, D195A, N198A, K200A, K205A, K209A, A210Q, A210S, A210G, P212A, P214A, E216A, K217A, S220A, K221A, A222T, K243A, Q245A, H251A, D259A, A261Q, E263A, E265A, V286A, S288A, K297A, S307A, E313A, H316A, N317A, H318A, Y319A (numbering corresponds to the sequence set forth in Uniprot reference number P01857-1).

In some embodiments, the linked domain has the sequence selected from the group consisting of SEQ ID Nos: 15, 16 or 17 or is at least 80%, at least 85%, at least 90%, at least 95%, at least 96%, at least 97%, at least 98%, or at least 99% identical to SEQ ID Nos: 15, 16 or 17.

### Transmembrane and Intracellular Domains

The transmembrane domain of a CAR bridges the extracellular portion (ectodomain) to the intracellular portion (endodomain) with its role being primarily structural. As such, the transmembrane domain can consist of any sequence that can anchor and span the lipid bilayer of a cell. However, the nature of the transmembrane domain can influence its localisation and expression.

In a preferred embodiment, the transmembrane domain has sequence identity to a sequence of a molecule involved in T cell synapse formation, or T cell signal induction. In some embodiments, the chimeric antigen receptor of the present invention includes a transmembrane domain which includes a sequence identical to all, or part of, the transmembrane domain of CD3, CD4, CD8 or CD28. In some embodiments, the transmembrane domain includes a sequence having identity to all, or part of, the transmembrane domain of CD8 or CD28. In some embodiments, the transmembrane domain has sequence identity to all, or part of, the transmembrane domain of CD28. In some embodiments, the transmembrane domain has the amino acid sequence identity to SEQ ID No. 18, or a functional variant thereof having at least 50%, 60%, 70%, 80%, 90%, 95%, 98%, 99% or 99.5% sequence identity.

In addition to the antigen recognition domain, the linker domain and the transmembrane domain, the chimeric antigen receptor of the present invention includes an intracellular (endo) domain which includes a signalling portion (a signalling domain).

In addition to the antigen recognition domain, the linker domain and the transmembrane domain, the chimeric antigen receptor of the present invention includes an intracellular (endo) domain which includes a signalling portion (the signalling domain).

The intracellular signalling domain of the chimeric antigen receptor can be any suitable domain that is capable of inducing, or participating in the induction of, an intracellular signalling cascade upon activation of the CAR as a result of recognition of an antigen by the antigen-recognition domain. The signalling domain of a CAR will be specifically chosen depending on the intended cellular outcome following activation of the CAR. Whilst there are many possible signalling domains, when used in immunotherapy and cancer therapy the signalling domains can be grouped into two general categories based on the receptor from which they are derived, namely activation receptors and co-stimulatory receptors (see further details below). Therefore, in some embodiments, the signalling domain includes a portion having an amino acid sequence identical to a signalling portion of an activation receptor, or a functional variant thereof. In some embodiments, the signalling domain includes a portion having an amino acid sequence identical to a signalling portion of a co-stimulatory receptor, or a functional variant.

As used throughout the specification the term “portion”, when used with respect to an activation receptor or co-stimulatory receptor, relates to any segment of the receptor that includes a sequence responsible for, or involved in, the initiation/induction of an intracellular signalling cascade following interaction of the receptor with its cognate antigen or ligand. An example of the initiation/induction of an intracellular signalling cascade for the T cell receptor (TCR) via CD3 is outlined below.

Whilst not wishing to be bound by theory, the extracellular portion of the TCR largely comprises heterodimers of either the clonotypic TCRα and TCRβ chains (the TCRα/β receptor) or the TCRγ and TCRδ chains (the TCRγδ receptor). These TCR heterodimers generally lack inherent signalling transduction capabilities and therefore they are non-covalently associated with multiple signal transducing subunits of CD3 (primarily CD3-zeta , -gamma, -delta, and -epsilon). Each of the gamma, delta, and epsilon chains of CD3 has an intracellular (cytoplasmic) portion that includes a single Immune-receptor-Tyrosine-based-Activation-Motif (ITAM), whilst the CD3-zeta chain includes three tandem ITAMs. Upon engagement of the TCR by its cognate antigen in the presence of MHC, and the association of a requisite co-receptor such as CD4 or CD8, signalling is initiated which results in a tyrosine kinase (namely Lck) phosphorylating the two tyrosine residues within the intracellular ITAM(s) of the CD3 chains. Subsequently, a second tyrosine kinase (ZAP-70-itself activated by Lck phosphorylation) is recruited to biphosphorylate the ITAMs. As a result, several downstream target proteins are activated which eventually leads to intracellular conformational changes, calcium mobilisation, and actin cytoskeleton re-arrangement that when combined ultimately lead to activation of transcription factors and induction of a T cell immune response.

As used throughout the specification the term “activation receptor” relates to receptors, or co-receptors that form a component of, or are involved in the formation of, the T cell receptor (TCR) complex, or receptors involved in the specific activation of immune cells as a result of recognition of an antigenic or other immunogenic stimulus.

Non-limiting examples of such activation receptors include components of the T cell receptor-CD3 complex (CD3-zeta, -gamma, -delta, and -epsilon), the CD4 co-receptor, the CD8 co-receptor, Fc receptors or Natural Killer (NK) cell associated activation receptors such a LY-49 (KLRA1), natural cytotoxicity receptors (NCR, preferably NKp46, NKp44, NKp30 or NKG2 or the CD94/NKG2 heterodimer). Consequently, in some embodiments of the CAR of the present invention the signalling domain includes a portion derived from any one or more of a member of the CD3 co-receptor complex (preferably at least a signalling portion of the CD3-Zeta (ζ) chain), the CD4 co-receptor, the CD8 co-receptor, a signalling portion of the Fc Receptor (FcR) (preferably a signalling portion of FcεRI or FcγRI) or NK associated receptors such a LY-49.

The specific intracellular signal transduction portion of each of the CD3 chains are known in the art. See for example WO/2022/104424, the entire content of which is incorporated herein by way of this reference, particularly with regard to linkers, transmembrane domains and intracellular domains of CARs.

In some embodiments of the present invention, the signalling domain includes a portion derived from, or having sequence homology to, CD3 (preferably the CD3-ζ chain or a portion thereof). In some embodiments, the signalling domain includes a sequence identical to all, or part of, the intracellular domain of CD3 zeta (CD3-ζ). In some embodiments, the portion of the CD3-ζco-receptor complex includes the amino acid sequence set forth in SEQ ID No. 19, or a functional variant thereof having at least 50%, 60%, 70%, 80%, 90%, 95%, 98%, 99% or 99.5% sequence identity.

Alternative signalling domains include intracellular portions of the Fc receptors, which are known in the art. For example, the intracellular portions of the FcεR1 or FcγRI receptors (see WO/2022/104424 for specific sequences). Various combinations of portions of activation receptors can be utilized to form the transmembrane (TM) and intracellular (IC) portions of the CAR for example the CD3ζ TM and CD3ζ IC (Landmeier S, et al. (2007). Gene-Engineered Varicella-Zoster Virus-Reactive CD4+ Cytotoxic T Cells Exert Tumor-Specific Effector Function, Cancer Res, 67, 8335-43; Guest R D, et al. (2005). The role of extracellular spacer regions in the optimal design of chimeric immune receptors: evaluation of four different scFvs and antigens, J Immunother, 28 (3), 203-211; Hombach A A, et al. (2007). T cell activation by antibody-like immunoreceptors: the position of the binding epitope within the target molecule determines the efficiency of activation of redirected T cells, J Immunol, 178, 4650-7; James S E, et al. (2008). Antigen sensitivity of CD22-specific chimeric TCR is modulated by target epitope distance from the cell membrane, J Immunol, 180 (10), 7028-38; Patel S D, et al. (1999). Impact of chimeric immune receptor extracellular protein domains on T cell function, Gene Ther, 6, 412-419; Haynes N M, et al. (2001). Redirecting Mouse CTL Against Colon Carcinoma: Superior Signaling Efficacy of Single-Chain Variable Domain Chimeras Containing TCR-ζ vs FcεRI-γ, J Immunol, 166, 182-1877; Annenkov A E, et al. (1998). Loss of Original Antigenic Specificity in T Cell Hybridomas Transduced with a Chimeric Receptor Containing Single-Chain Fv of an Anti-Collagen Antibody and FcεRI-Signaling γ Subunit, J Immunol, 161, 6604-6613).

As discussed above, in some embodiments of the chimeric antigen receptor of the present invention the signalling domain includes a portion having an amino acid sequence identical to a signalling portion of a co-stimulatory receptor.

As used throughout the specification the term “co-stimulatory receptor” relates to receptors or co-receptors that assist in the activation of an immune cell upon antigen specific inducement of an activation receptor. As will be understood, co-stimulatory receptors do not require the presence of antigen and are not antigen specific, but are typically one of two signals, the other being an activation signal, which is required for the induction of an immune cellular response. In the context of an immune response a co-stimulation receptor is typically activated by the presence of its expressed ligand on the surface of an antigen-presenting cell (APC) such as a dendritic cell or macrophage. With specific regard to T cells, co-stimulation is necessary to lead to cellular activation, proliferation, differentiation, and survival (all of which are generally referred to under the umbrella of T cell activation), whilst presentation of an antigen to a T cell in the absence of co-stimulation can lead to anergy, clonal deletion and/or the development of antigen specific tolerance. Importantly, co-stimulatory molecules can inform the T cell response to a simultaneously encountered antigen. Generally, an antigen encountered in the context of a ‘positive’ co-stimulatory molecule will lead to activation of the T cell and a cellular immune response aimed at eliminating cells expressing that antigen. Whilst an antigen encountered in the context of a ‘negative’ co-receptor will lead to an induced state of tolerance to the co-encountered antigen.

Non-limiting examples of T cell co-stimulatory receptors include CD27, CD28, CD30, CD40, DAP10, OX40, 4-1BB (CD137), ICOS. Specifically, CD27, CD28, CD30, CD40, DAP10, OX40, 4-1BB (CD137), and ICOS all represent ‘positive’ co-stimulatory molecules that enhance activation of a T cell response. Accordingly, in some embodiments of the first aspect of the present invention, the signalling domain includes a portion derived from any one or more of CD27, CD28, CD30, CD40, DAP10, OX40, 4-1BB (CD137) and ICOS.

In some embodiments of the present invention, the signalling domain includes a portion derived from the CD28, OX40 or 4-1BB co-stimulatory receptors. In some embodiments, the signalling domain includes a portion of 4-1BB as set out in SEQ ID No: 20, or a functional variant thereof having at least 50%, 60%, 70%, 80%, 90%, 95%, 98%, 99% or 99.5% sequence identity.

Various portions of co-stimulatory receptors can be utilized to form the transmembrane (TM) and intracellular (IC) portions of the CAR, alone or in combination. Examples of combinations include the CD8 TM and DAP10 IC or CD8 TM and 4-1BB IC (Marin V. et al. Exp Hematol., 2007; 35:1388-97), the CD28 TM and the CD28 IC (Wilkie S. et al. J Immunol., 2008; 180:4901-9; Maher J. et al. Nat Biotechnol., 2002; 20:70-5), and the CD8 TM and the CD28 IC (Marin V. et al. Exp Hematol., 2007; 35:1388-97).

Sequence information for the above-referenced activation and co-stimulatory receptors is readily accessible in a variety of databases. For example, embodiments of human amino acid, gene and mRNA sequences for these receptors are provided in Table 3.

TABLE 3
Summary of Activation and Co-stimulation
Receptor Sequence Information

| | Uniprot | NCBI Gene | GeneBank mRNA |
|---|---|---|---|
| Receptor Name | Ref No. | ID No. | Ref No. |
| CD3-zeta | P20963 | 919 | GI: 166362721 |
| CD3-gamma | P09693 | 917 | GI: 166362738 |
| CD3-delta | P04234 | 915 | GI: 98985799 |
| CD3-epsilon | P07766 | 916 | GI: 166362733 |
| CD4 | P0173 | 920 | GI: 303522473 |
| CD8 alpha | P01732 | 925 | GI: 225007534 |
| CD8 beta | P01966 | 926 | GI: 296010927 |
| FcγRI | P12314 | 2209 | GI: 31331 |
| FcεR1 | Q01362 | 2206 | GI: 219881 |
| Ly-49 (KLRAI) | Q7Z556 | 10748 | GI: 33114184 |
| NKp46 | O76036 | 9437 | GI: 3647268 |
| NKp44 | O95944 | 9436 | GI: 4493701 |
| NKp30 | O14931 | 259197 | GI: 5823969 |
| CD94 | Q13241 | 3824 | GI: 1098616 |
| CD27 | P26842 | 939 | GI: 180084 |
| CD28 | P10747 | 940 | GI: 338444 |
| CD30 | P28908 | 943 | GI: 180095 |
| CD40 | P25942 | 958 | GI: 29850 |
| DAP10 | Q9UBK5 | 10870 | GI: 5738198 |
| OX40 | P43489 | 7293 | GI: 472957 |
| 4-1BB (TNR9) | Q07011 | 3604 | GI: 571320 |
| ICOS | Q9Y6W8 | 29851 | GI: 9968295 |
| CTLA-4 | P16410 | 1493 | GI: 291928 |
| PD-1 | Q15116 | 5133 | GI: 2149002 |

Whilst Table 3 is provided with reference to human activation and co-stimulatory receptors, it would be understood by a person skilled in the art that homologous and orthologous versions of each receptor are present in the majority of mammalian and vertebrate species. Therefore, the above-referenced sequences are only provided as non-limiting examples of receptor sequences that may be included in a CAR of the first aspect of the present invention and homologous and orthologous sequences from any desired species may be used to generate a CAR that is suitable for the given species.

In some embodiments of the invention, the transmembrane domain, and a portion of the signalling domain share homology with the same molecule. For example, a portion of CD3 including the transmembrane domain and a signalling domain may be utilised. In some embodiments, the transmembrane domain includes, or consists of, a sequence identical to all or a portion of the transmembrane domain of CD28 and the signalling domain includes, or consists of, a sequence identical to all or a portion of the intracellular domain of CD28.

In some embodiments of the present invention, the signalling domain includes a portion derived from an activation receptor and a portion derived from a co-stimulatory receptor. Whilst not wishing to be bound by theory, in this context the recognition of an antigen by the antigen-recognition domain of the CAR will simultaneously induce both an intracellular activation signal and an intracellular co-stimulatory signal. Consequently, this will simulate the presentation of an antigen by an APC expressing co-stimulatory ligand. Alternatively, the CAR could have a signalling domain that includes a portion derived from either an activation receptor or a co-stimulatory receptor. In this alternative form, the CAR will only induce either an activating intracellular signalling cascade or a co-stimulatory intracellular signalling cascade.

In some embodiments of the invention the signalling domain includes, or consists of, a sequence identical to all or a portion of the intracellular domain of 4-1BB and CD3-ζ chain.

In some embodiments, the CAR will have a signalling domain that includes a portion of a single activation receptor and portions of multiple co-stimulatory receptors. In some embodiments, the CAR will have a signalling domain including a sequence identical to portions of multiple activation receptors and a portion derived from a single co-stimulatory receptor. In some embodiments, the CAR will have a signalling domain that includes a sequence identical to portions of multiple activation receptors and portions of multiple co-stimulatory receptors. In some embodiments, the CAR will have a signalling domain that includes a sequence identical to a portion of a single activation receptor and portions of two co-stimulatory receptors. In some embodiments, the CAR will have a signalling domain that includes a sequence identical to a portion of a single activation receptor and portions derived from three co-stimulatory receptors. In some embodiments, the CAR will have a signalling domain that includes a sequence identical to portions of two activation receptors, and a portion of one co-stimulatory receptor. In some embodiments, the CAR will have a signalling domain that includes a sequence identical to portions of two activation receptors and portions of two co-stimulatory receptors. As will be understood there are further variations of the number of activation receptors and co-stimulatory receptors, and the above examples are not considered to be limiting on the possible combinations included herein.

In some embodiments of the invention, the sequence of the transmembrane domain and at least a portion of the signalling domain have sequence similarity to portions of distinct molecules. In some embodiments, the transmembrane domain includes, or consists of, a sequence identical to all or a portion of the transmembrane domain of CD28 and the signalling domain includes, or consists of, a sequence identical to all or a portion of the intracellular domain of 4-1BB and CD3-ζ chain.

CAR are currently referred to as 1^{st} generation to 5^{th} generation (see Labanieh L and Mackall C L. (2023), CAR immune cells: design principles, resistance, and the next generation. Nature, 614 (7949): pg635-648; and Zheng Z, et al. (2023). Fine-Tuning through Generations: Advances in Structure and Production of CAR-T Therapy. Cancers (Basel). 3; 15 (13): 3476.—the entire disclosures of which are incorporated herein). In some embodiments, the CAR of the present invention is a 3rd generation CAR or later (i.e., contains an activation domain and two or more co-stimulatory domains). In some embodiments, the CAR is a 4th generation CAR or later (i.e., a TRUCK—T cell redirected for universal cytokine-mediated killing). In some embodiments, the CAR T cell includes a termination receptor to permit removal of CAR T cells after administration).

### Chimeric Antigen Receptor

Exemplified chimeric Antigen Receptors (CARs) of the present invention were prepared using two scFv fusion proteins having two orientations of the variable light and variable heavy domains of the MIL-38 antibody (WO2016/168885A1; and Truong Q, et al. (2016). Glypican-1 as a Biomarker for Prostate Cancer: Isolation and Characterization. J Cancer. May 21; 7 (8): 1002-9.)

As illustrated in FIGS. 15A to 15F, two scFv domains were prepared with the following orientations:

1. light chain variable region (2)—Whitlow linker (3) (PMID: 8309948)—heavy chain variable region (4) (denoted by the code CNA500xxx); and

2. heavy chain variable region (4)—Whitlow linker (3)—light chain variable region (2) (denoted by the code CNA510xxx).

Further, three different linker domains were utilised namely:

$$\begin{matrix}Linker 1 & \\ IgG 4 hinge ( 5 ) ( SEQ ID No : 15 ) - ( denoted by the code CNA 5 x 02 xx ) ; & \end{matrix}$$

$$\begin{matrix}Linker 2 & \\ IgG 4 hinge + IgG 4 CH 3 ( 11 ) ( SEQ ID No : 16 ) - ( denoted by the code CNA 5 x 03 xx ) ; & \end{matrix}$$

$$and$$

$$\begin{matrix}Linker 3 & \\ IgG hinge + IgG 4 CH 2 L 235 D and N 297 Q mutations + IgG 4 CH 3 ( 12 ) ( SEQ ID No : 17 ) - ( denoted by the code CNA 5 x 04 xx ) . & \end{matrix}$$

The sequences (SEQ ID NOs) of the chimeric antigen receptor and component parts are provided in Table 4.

TABLE 4
Summary of sequence identifiers

| | Sequence or |
|---|---|
| Component | SEQ ID NO: |
| Light chain variable region (a.a.) | SEQ ID NO: 1 |
| Heavy chain variable region (a.a.) | SEQ ID NO: 2 |
| Light chain CDR1 (a.a.) | SEQ ID NO: 3 |
| Light chain CDR2 (a.a.) | TAKTLAD |
| Light chain CDR3 (a.a.) | QHFWSNPWT |
| Heavy chain CDR1 (a.a.) | DYSMN |
| Heavy chain CDR2 (a.a.) | SEQ ID NO: 4 |
| Heavy chain CDR3 (a.a.) | HYDYGGFPY |
| scFv: leader - light to heavy orientation (DNA) | SEQ ID NO: 5 |
| scFv: leader - heavy to light orientation (DNA) | SEQ ID NO: 6 |
| CNA500200 complete vector sequence (DNA) | SEQ ID NO: 7 |
| CNA500300 complete vector sequence (DNA) | SEQ ID NO: 8 |
| CNA500400 complete vector sequence (DNA) | SEQ ID NO: 9 |
| CNA 510200 complete vector sequence (DNA) | SEQ ID NO: 10 |
| CNA 510300 complete vector sequence (DNA) | SEQ ID NO: 11 |
| CNA 510400 complete vector sequence (DNA) | SEQ ID NO: 12 |
| scFv: leader - light to heavy orientation (a.a.) | SEQ ID NO: 13 |
| scFv: leader - heavy to light orientation (a.a.) | SEQ ID NO: 14 |
| Linker 1 - IgG4 hinge | SEQ ID NO: 15 |
| Linker 2 - IgG4 hinge + IgG4 CH3 | SEQ ID NO: 16 |
| Linker 3 - IgG hinge + IgG4 CH2 L235D and N297Q | SEQ ID NO: 17 |
| mutations + IgG4 CH3 | |
| CD28 transmembrane domain | SEQ ID NO: 18 |
| CD3 zeta | SEQ ID NO: 19 |
| 4-1BB | SEQ ID NO: 20 |
| CNA500200 construct sequence (a.a.) | SEQ ID NO: 21 |
| CNA500300 construct sequence (a.a.) | SEQ ID NO: 22 |
| CNA500400 construct sequence (a.a.) | SEQ ID NO: 23 |
| CNA 510200 construct sequence (a.a.) | SEQ ID NO: 24 |
| CNA 510300 construct sequence (a.a.) | SEQ ID NO: 25 |
| CNA 510400 construct sequence (a.a.) | SEQ ID NO: 26 |
| MIL-38 leader | SEQ ID NO: 27 |
| scFv: leader - light to heavy orientation (a.a) | SEQ ID NO: 28 |
| scFv: leader - heavy to light orientation (a.a) | SEQ ID NO: 29 |

With further reference to FIGS. 15A to 15F, the CARs exemplified in the present invention as specific embodiments, include the components of: the MIL-38 leader sequence (1), a transmembrane region having sequence identity to a portion of CD28 (6), a costimulatory domain having sequence identity to a portion of 4-1BB (7), and an activation domain having sequence identity to a portion of CD3 zeta (8). The exemplified CARs also included a truncated ECF receptor (EGFRt) (10) which allowed for analysis of transduction and expression in cells. The EGFRt was connect by way of the self-cleavage site T2A (9) allowing separation of EGFRt from the CAR. The sequences of EGFRt and T2a are known in the art and published in WO/2022/104424.

In some embodiments, the CAR will include an antigen recognition domain specific for GPC1, a linker domain having sequence identity to the IgG4 hinge region, a transmembrane region having sequence identity to the CD28 transmembrane sequence, an intracellular portion having sequence identity to a signalling region of 4-1BB and/or an intracellular portion having sequence identity to a signalling portion of CD3zeta, or functional variant of the described portions, domains or regions.

In some embodiments, the CAR will include an antigen recognition domain specific for GPC1, a linker domain having sequence identity to the IgG4 hinge region combined with the IgG4 CH3 region, a transmembrane region having sequence identity to the CD28 transmembrane sequence, an intracellular portion having sequence identity to a signalling region of 4-1BB and/or an intracellular portion having sequence identity to a signalling portion of CD3zeta, or functional variants of the described portions, domains or regions.

In some embodiments, the CAR will include an antigen recognition domain specific for GPC1, a linker domain having sequence identity to the IgG4 hinge region combined with the IgG4 CH2 region (which may include L235D and N297Q mutations) and the IgG4 CH3 region, a transmembrane region having sequence identity to the CD28 transmembrane sequence, an intracellular portion having sequence identity to a signalling region of 4-1BB and/or an intracellular portion having sequence identity to a signalling portion of CD3zeta, or functional variants of the described portions, domains or regions.

In some embodiments of invention, the chimeric antigen receptor includes, or consists of, an amino acid sequence selected from the group consisting of: SEQ ID Nos. 21, 22, 23, 24, 25 and 26 (CNA500200, CNA500300, CNA500400, CNA510200, CNA510300 and CNA510400), or functional variants thereof.

As would be understood by a person skilled in the art modification of the CAR receptors described herein can be made without deviating from the scope of the present invention. For example, with respect to SEQ ID Nos Nos: 21, 22, 23, 24, 25 and 26 the preferred function of the CAR is to recognise GPC1 and induce an intracellular signal which results in the activation of a T cell expressing the CAR. Accordingly, variation to portions of the amino acid sequence of the chimeric antigen receptor may be made without significantly altering the specificity of the CAR and/or activation of a cell (such as a T cell) expressing the CAR. Such variations may include, but are not limited to, variations in the hinge region of the chimeric antigen receptor, variations in the transmembrane domain, and variations in the portions of the activation receptors and/or co-stimulatory receptors that comprise the intracellular domain of the chimeric antigen receptor. When undertaking such variations, a person skilled in the art will utilise the knowledge and skills with the intention to arrive at a workable CAR. As such, the scope of the variations excludes those which are immediately recognisable to a person skilled in the art as resulting in the abrogation of the function of the CAR.

In some embodiments of the invention, the chimeric antigen receptor includes, or consists of, a variant of SEQ ID Nos. 21, 22, 23, 24, 25 or 26 having an amino acid sequence having at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 98.2%, at least 98.4%, at least 98.6%, at least 98.8%, at least 99%, at least 99.1%, at least 99.2%, at least 99.3%, at least 99.4%, at least 99.5%, at least 99.6%, at least 99.7%, at least 99.8% or at least 99.9% sequence identity to an amino acids selected from the group consisting of SEQ ID Nos. 21, 22, 23, 24, 25 and 26

### Nucleic Acid Constructs and Genetic Modification of Cells

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

As such, the present invention further provides a nucleic acid molecule, or a nucleic acid construct including a nucleic acid molecule, having a nucleic acid sequence encoding the chimeric antigen receptor described above.

Further, the nucleic acid construct may be an expression vector including a nucleic acid sequence encoding the chimeric antigen receptor described above.

In some embodiments, the nucleic acid molecule includes a nucleotide sequence which encodes an amino acid sequence selected from the group consisting of SEQ ID Nos: 21, 22, 23, 24, 25 and 26, or a variant of these sequences as previously defined.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus, the term “nucleic acid molecule” embraces chemically, enzymatically, or metabolically modified forms.

In some embodiments of the invention, the nucleic acid molecule includes the portion of the nucleotide sequence set forth in SEQ ID Nos: 7, 8, 9, 10, 11 or 12, encoding for the amino acids set forth in of SEQ ID Nos. 21, 22, 23, 24, 25 and 26, or a functional variant thereof.

To absolve doubt, it is to be understood that functional variants of the relevant portions of SEQ ID Nos: 7, 8, 9, 10, 11 or 12 includes sequence variants having one or more different nucleic acids, but which still encode identical amino acid sequences. Because of the degeneracy of the genetic code, a large number of nucleic acids can encode any given protein. For instance, the codons GCA, GCC, GCG and GCU all encode the amino acid alanine. One of skill in the art will recognise that each codon in a nucleic acid sequence (except AUG, which is ordinarily the only codon for methionine, and TGG, which is ordinarily the only codon for tryptophan) can be modified to yield a functionally identical molecule. Accordingly, each silent variation of a nucleotide sequence that encodes a polypeptide is implicit in each described sequence.

It is to be understood that a nucleic acid construct, in accordance with the invention, may further comprise one or more of: an origin of replication for one or more hosts; a selectable marker gene which is active in one or more hosts; and/or one or more transcriptional control sequences, wherein expression of the nucleic acid molecule is under the control of a transcriptional control sequence.

As used herein, the term “selectable marker gene” includes any gene that confers a phenotype on a cell in which it is expressed, to facilitate the identification and/or selection of cells, which are transfected or transduced with the construct.

“Selectable marker genes” include any nucleotide sequences which, when expressed by a cell transduced with the construct, confer a phenotype on the cell that facilitates the identification and/or selection of these transduced cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine (TK) kinase gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance gene; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS). Further, cell selection markers for T cells are specifically discussed in Barese, C. N. and Dunubar C. E., Hum. Gene Ther., 2011; 22 (6): pp. 659-68. These markers include neomycin (NEO) resistance genes, ANGFR (non-signalling NGFR), truncated CD34 and truncated non-signalling CD19 (ACD19). Embodiments of the present invention (as described further herein) utilise a truncated form of the epithelial growth factor receptor (EGFRt). Further techniques have been developed for tracking CAR T cells in vivo including modified eDHFD (see Sellmyer, M. A. et al. Mol. Ther., 2020; 28 (1): pp. 42-51).

Furthermore, it should be noted that the selectable marker gene may be a distinct open reading frame in the construct or may be expressed as a fusion protein with another polypeptide (e.g., the CAR).

As set out above, the nucleic acid construct may also comprise one or more transcriptional control sequences. The term “transcriptional control sequence” should be understood to include any nucleic acid sequence which effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid which confers, activates, or enhances expression of a nucleic acid in a cell.

In some embodiments, at least one transcriptional control sequence is operably connected to the nucleic acid molecule of the second aspect of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter which is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (E3A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: tetracycline regulated promoters (e.g., see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence which is operable in the cells, tissues, or organs in which it is intended to be used. Suitable terminators would be known to a person skilled in the art.

As will be understood, the nucleic acid construct in accordance with the invention can further include additional sequences, for example sequences that permit enhanced expression, cytoplasmic or membrane transportation, and location signals. Specific non-limiting examples include an Internal Ribosome Entry Site (IRES), an N-terminal interleukin-2 signal peptide (Moot R. et al., Mol Ther Oncolytics, 2016; 3:16026), CSF2RA, IgE leader sequence (WO2017147458), influenza hemagglutinin signal sequence (Quitterer, U. et al., Biochem. Biophys. Res., 2011: 409 (3): pp. 544-579) amongst others. A review of signal peptides is provided in Owki, H. et al. Eur. J. Cell Biol., 2018; 97 (6): pp. 422-441, which is herein incorporated by reference.

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

The nucleic acid construct may be in any suitable form, such as in the form of a plasmid, phage, transposon, cosmid, chromosome, vector, etc., which is capable of replication when associated with the proper control elements and which can transfer gene sequences, contained within the construct, between cells.

Thus, the term vector includes cloning and expression vehicles, as well as viral vectors. In some embodiments, the nucleic acid construct is a vector. In some embodiments, the vector is a viral vector, and therefore the present invention provides a viral vector including a nucleic acid molecule, or the nucleic acid construct, which encode the CAR described above. In some embodiments, the vector is a DNA vector or mRNA vector.

In at least some embodiments, the present invention provides a nucleic acid molecule, or a nucleic acid construct, encoding the CAR described above, for use in preparing a genetically modified cell. Further, in at least some embodiments, the present invention provides a use of a nucleic acid molecule in the preparation of a vector for the transformation, transfection, or transduction of a cell such as those described herein. Cells suitable for genetic modification can be heterologous or autologous.

In some embodiments, the cell is used in a method, or in the preparation of a medicament, for the prevention or treatment of cancer. Consequently, in some embodiments, the present invention provides the use of a vector in the preparation of a medicament for the prevention or treatment of cancer, in particular ovarian cancer expressing Glypican-1.

Methods are known in the art for the deliberate introduction (transfection/transduction) of exogenous genetic material, such as the nucleic acid construct, into eukaryotic cells. As will be understood the method best suited for introducing the nucleic acid construct into the desired host cell is dependent on many factors, such as the size of the nucleic acid construct, the type of host cell the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation (see Potter and Heller. “Transfection by Electroporation.” Curr. Prot. Mol. Bio., ed. Frederick M. Ausubel et al. 2003: Unit-9.3), sonoporations (Wang, M et al. Sci. Reps., 2018; 8:3885), heat-shock or optical transfection; particle-based methods such as ‘gene gun’ delivery, magnetofection, or impalefection, lipid nanoparticles or viral transduction.

A variety of viral transduction techniques for mammalian cells are known in the art. Common viral vectors include lentivirus and retrovirus. An exemplary protocol is provided in Wang L et al., Proc. Natl. Acad. Sci., 2011; 108: E803-12. Alternative viral vectors include, HSV, Adenovirus and AAV (Howarth J et al. Cell. Bio. & Toxic., 2010, vol. 26, issue 1, pp 1-20).

In some embodiments, the present invention provides a lentivirus comprising a nucleic acid encoding a chimeric antigen receptor as described herein. Further, the present invention provides a use of a viral vector, preferably a retrovirus such as a lentivirus or a gamma retrovirus, in the preparation of a genetically modified cell or a medicament for the prevention or treatment of cancer, or for the killing of a cell, expressing Glypican-1 or aberrantly expressing Glypican-1.

The transduction of cells can result in genomic integration of DNA encoding the CAR described above. Alternatively, the DNA can be transiently expressed within the transduced cell. Each of these has positives and negatives. Genomic integrated DNA is stably expressed and replicated to progeny cells during cell replication. This ensures a robust immune response and a significant increase in CAR-expressing T cells in vivo.

Alternatively, transient transduction (often achieved by transducing with mRNA) leads to temporary CAR expression in cells. This normally leads to a much lower response but provides more control to the practitioner to increase or decrease the “dosage” as needed.

As described above, in some embodiments, the invention provides the use of a DNA vector, or recombinant DNA, in the preparation of a viral vector for the genetic transduction of a cell. The cell can be any cell, however suitable examples are provided.

The nucleic acid construct will be selected depending on the desired method of transfection/transduction. In some embodiments, the nucleic acid construct is a viral vector, and the method for introducing the nucleic acid construct into a host cell is viral transduction. Methods are known in the art for utilising viral transduction to elicit expression of a CAR in a PBMC such as a T cell (Parker, L L. et al. Hum Gene Ther. 2000; 11:2377-87) and more generally utilising retroviral systems for transduction of mammalian cells (Cepko, C. and Pear, W. Curr Protoc Mol Biol. 2001, unit 9.9). In some embodiments, the nucleic acid construct is a plasmid, a cosmid, an artificial chromosome or the like, and can be transfected into the cell by any suitable method known in the art.

Techniques are known in the art for selection/isolation of cell subsets. These include Fluorescent Activated Cell Sorting (Basu S. et al. J. Vis. Exp. 2010; 41:1546), techniques utilising antibodies immobilised on a substrate, such as magnetic cell isolation (MACS®) device to immunomagnetically select cells expressing the desired markers (Zola H. et al. Blood, 2005; 106 (9): 3123-6), or use of microfluidic chips. A series of cell markers can be used to isolate cells of the immune system including (but not limited to), BCR, CCR10, CD1a, CD1b, CD1c, CD1d, CD3, CD4, CD5, CD7, CD8, CD10, CD11b, CD11c, CD13, CD16, CD19, CD21, CD23, CD25, CD27, CD31, CD32, CD33, CD34, CD38, CD39, CD40, CD43, CD45, CD45RA, CD45RO, CD48, CD49d, CD49f, CD51, CD56, CD57, CD62, CD62L, CD68, CD69, CD62, CD62L, CD66b, CD68, CD69, CD73, CD78, CD79a, CD79b, CD80, CD81, CD83, CD84, CD85g, CD86, CD94, CD103 CD106, CD115, CD117, CD122, CD123, CD126, CD127, CD130, CD138, CD140a, CD140b, CD141, CD152, CD159a, CD160, CD161, CD163, CD165, CD169, CD177, CD178, CD183, CD185, CD192, CD193, CD194, CD195, CD196, CD198, CD200, CD200R, CD203c, CD205, CD206, CD207, CD209, CD212, CD217, CD218 alpha, CD229, CD244, CD268, CD278, CD279, CD282, CD284, CD289, CD294, CD303, CD304, CD314, CD319, CD324, CD335, CD336, CXCR3, Dectin-1, Tc epsilor R1 alpha, Flt3, Granzyme A, Granzyme B, IL-9, IL-13apha1, IL-21R, iNOS, KLRG1, MARCO, MHC class II, RAG, ROR Gamma T, Singlec-8, ST2, TCR alpha/beta, TCR gamma/delta, TLR4, TLR7, VEGF, ZAP70

Of particular note are the T cell markers CCR10, CD1a, CD1c, CD1d, CD2, CD3, CD4, CD5, CD7, CD8, CD9, CD10, CD11b, CD11c, CD13, CD16, CD23, CD25, CD27, CD31, CD34, CD38, CD39, CD43, CD45, CD45RA, CD45RO, CD48, CD49d, CD56, CD62, CD62L, CD68, CD69, CD73, CD79a, CD80, CD81, CD83, CD84, CD86, CD94, CD103, CD122, CD126, CD127, CD130, CD140a, CD140b, CD152, CD159a, CD160, CD161, CD165, CD178, CD183, CD185, CD192, CD193, CD194, CD195, CD196, CD198, CD200, CD200R, CD212, CD217, CD218 alpha, CD229, CD244, CD278, CD279, CD294, CD304, CD314, CXCR3, Flt3, Granzyme A, Granzyme B, IL-9, IL-13alpha1, IL-21R, KLRG1, MHC class II, RAG, ROR gamma T, ST2, TCR alpha/beta, TCR gamma/delta, ZAP70. Particularly preferred cell markers for T cell selection include TCRgamma, TCR delta, CD3, CD4 and CD8.

Isolated cells can then be cultured to modify cell activity, expanded, or activated. Techniques are known in the art for expanding and activating cells (Wang X. and Rivière I. Mol. Thera. Oncolytics. 2016; 3:16015). These include; using anti-CD3/CD28 microbeads (Miltenyi Biotec or Thermofisher Scientific—as per manufacturer's instructions), or other forms of immobilised CD3/CD28 activating antibodies. Activated/genetically modified cells can then be expanded in vitro in the presence of cytokines (such as with IL-2, IL-12, IL-15, or IL-17) and then cryopreserved. An overview of methods for expanding CAR T cells is provided in Wang and Rivièra ibid).

The present invention further provides a genetically modified cell including the chimeric antigen receptor, nucleic acid molecule, or nucleic acid construct as described above. In some embodiments, the genetically modified cell includes a genomically integrated form of the nucleic acid molecule or construct. In some embodiments, the genetically modified cell is a leukocyte. In some embodiments, the genetically modified cell is a Peripheral Blood Mononuclear Cell (PBMC). In some embodiments, the genetically modified cell is a myeloid cell. In some embodiments, the genetically modified cell is a monocyte. In some embodiments, the genetically modified cell is a macrophage. In some embodiments, the genetically modified cell is a lymphocyte. In some embodiments, the genetically modified cell is a T cell. In some embodiments, the genetically modified cell is an alpha beta (αβ) T cell. In some embodiments, the genetically modified cell is a gamma delta (γδ) T cell. In some embodiments, the genetically modified cell is a CD3+ T cell (such as a naive CD3+ T cells or a memory CD3+ T cell). In some embodiments, the T cell is a CD4+ T cell (such as a naive CD4+ T cells or a memory CD4+ T cell). In some embodiments, the T cell is a CD8+ T cell (such as a naive CD8+ T cells or a memory CD8+ T cell). In some embodiments, the genetically modified cell is a natural killer cell. In some embodiments, the genetically modified cell is a natural killer T (NKT) cell.

### Uses of CAR to Treat or Prevent Cancer

In addition to the identification by the present inventors of high expression of Glypican-1 is ovarian cancer, high expression of Glypican-1 has been reported in pancreatic ductal adenocarcinoma, breast cancer, cervical cancer, lung cancer, malignant pleural mesothelioma, and glioblastoma. (see Nishigaki T, et al. (2020). Anti-glypican-1 antibody-drug conjugate is a potential therapy against pancreatic cancer. Br J Cancer, 122, 1333-41; Duan L, et al. (2013). GPC-1 may serve as a predictor of perineural invasion and a prognosticator of survival in pancreatic cancer. Asian J Surg, 36, 7-12; Matsuda K, et al. (2001). Glypican-1 is overexpressed in human breast cancer and modulates the mitogenic effects of multiple heparin-binding growth factors in breast cancer cells. Cancer Res, 61, 5562-9; Matsuzaki S, et al. (2018). Anti-glypican-1 antibody-drug conjugate exhibits potent preclinical antitumor activity against glypican-1-positive uterine cervical cancer. Int J Cancer, 142, 1056-66; Chiu K, et al. (2018). Glypican-1 immunohistochemistry does not separate mesothelioma from pulmonary adenocarcinoma. Mod Pathol, 31, 1400-3; and Saito T, et al. (2017). High expression of glypican-1 predicts dissemination and poor prognosis in glioblastomas. World Neurosurg, 105, 282-8).

Accordingly, the CAR of the present invention can be used for treating or preventing any cancer associated with Glypican-1 expression, including (but not limited to) pancreatic ductal adenocarcinoma, breast cancer, cervical cancer, lung cancer, malignant pleural mesothelioma, glioblastoma, and ovarian cancer. A particularly envisaged embodiment of the method of treating or preventing cancer is a method of treating or preventing ovarian cancer in a subject, the method comprising administering an anti-GPC1 CAR cell to the subject.

The present invention also provides a pharmaceutical composition including a genetically modified cell including a chimeric antigen receptor, a nucleic acid molecule or a nucleic acid construct as described above and one or more of a pharmaceutically acceptable carrier, excipient or diluent, wherein the pharmaceutical composition is used in the prevention or treatment of cancer, including (but not limited to) pancreatic ductal adenocarcinoma, breast cancer, cervical cancer, lung cancer, malignant pleural mesothelioma, glioblastoma, and ovarian cancer. In a preferred embodiment, the cancer is ovarian cancer.

### Methods of Diagnosis and Prognosis

Also provided is a method of diagnosing or assessing the prognosis of a subject having ovarian cancer, the method comprising determining the level of Glypican-1 in ovarian cells, or suspected cancer cells, from the subject, wherein an elevated level of Glypican-1 indicates the presence of ovarian cancer and/or indicates a worse prognosis.

In some embodiments, the ovarian cancer is categorised by FIGO's system and includes ovarian cancer, fallopian cancer, or peritoneal cancer (see Kehoe, S and Bhatla, N, FIGO cancer report 2021, International Journal of Gynecology & Obstetrics).

“Elevated expression of Glypican-1” as used herein means an increase in Glypican-1 mRNA or protein relative to a control value. In some embodiments, the control value is normal expression. In some embodiments, the control value is a threshold value. In some embodiments, the control value is a precancer value. In some embodiments where the ovarian cancer is relapsed ovarian cancer, the control value is a value prior to relapse.

In some embodiments, the normal expression is determined from non-cancerous ovarian cells, or—in the case of some ovarian cancers—fallopian cells. In some embodiments, these cells are of the same sort of cells as the cancer cells, for example epithelial cells.

In some embodiments, the threshold value is a predetermined threshold value. Such predetermined threshold values may be based on previous analysis of the subject, or may be based on a population value such as a median or mean value determined from multiple samples of non-cancerous or healthy cells from comparable members of the population.

In some embodiments, the expression of Glypican-1 is elevated by at least, or at least about, 5%, or 10%, or 15%, or 20%, or 25%, or 30%, or 35%, or 40%, or 45%, or 50%, or 55%, or 60%, or 65%, or 70%, or 75%, or 80%, or 85%, or 90%, or 95% or 100%, 120%, 140%, 160%, 180%, 200%, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, or 10 fold.

In some embodiments, a worse prognosis means a lower overall survival rate or a lower rate of progression-free survival of the subject. In some embodiments, elevated gene expression indicates lower overall survival. In some embodiments, elevated protein expression indicates lower overall survival. In some embodiments, elevated gene expression and elevated protein indicates lower overall survival. In some embodiments, elevated gene expression indicates lower progression-free survival. In some embodiments, elevated protein expression indicates lower progression-free survival. In some embodiments, elevated gene expression and elevated protein indicates lower progression-free survival.

In some embodiments, a worse prognosis means a quicker progression of cancer or more rapid growth of a tumour. In some embodiments, a worse prognosis means a higher likelihood of progressing to a higher stage of cancer. In some embodiments, a worse prognosis means a higher likelihood of metastasis of the primary cancer.

Stages of ovarian cancer (including fallopian and peritoneal cancer) are provided in Table 5.

TABLE 5
FIGO stages of ovarian, fallopian, and peritoneal cancer.

| | |
|---|---|
| Stage I: Tumor confined to ovaries or fallopian tube(s) | T1-N0-M0 |
| IA: Tumor limited to 1 ovary (capsule intact) or fallopian tube; no | T1a-N0-M0 |
| tumor on ovarian or fallopian tube surface; no malignant cells in | |
| the ascites or peritoneal washings | |
| IB: Tumor limited to both ovaries (capsules intact) or fallopian | T1b-N0-M0 |
| tubes; no tumor on ovarian or fallopian tube surface; no malignant | |
| cells in the ascites or peritoneal washings | |
| IC: Tumor limited to 1 or both ovaries or fallopian tubes, with any | |
| of the following: | |
| IC1: Surgical spill | T1c1-N0-M0 |
| IC2: Capsule ruptured before surgery or tumor on ovarian or | T1c2-N0-M0 |
| fallopian tube surface | |
| IC3: Malignant cells in the ascites or peritoneal washings | T1c3-N0-M0 |
| Stage II: Tumor involves 1 or both ovaries or fallopian tubes | T2-N0-M0 |
| with pelvic extension (below pelvic brim) or peritoneal | |
| cancer | |
| IIA: Extension and/or implants on uterus and/or fallopian tubes | T2a-N0-M0 |
| and/or ovaries | |
| IIB: Extension to other pelvic intraperitoneal tissues | T2b-N0-M0 |
| Stage III: Tumor involves 1 or both ovaries or fallopian tubes, | T1-3/N0-1/M0 |
| or peritoneal cancer, with cytologically or histologically | |
| confirmed spread to the peritoneum outside the pelvis and/or | |
| metastasis to the retroperitoneal lymph nodes | |
| IIIA1: Positive retroperitoneal lymph nodes only (cytologically or | T1/T2-N1-M0 |
| histologically proven): | |
| IIIA1(i) Metastasis up to 10 mm in greatest dimension | |
| IIIA1(ii) Metastasis more than 10 mm in greatest dimension | |
| IIIA2: Microscopic extrapelvic (above the pelvic brim) peritoneal | T3a2-N0/N1-M0 |
| involvement with or without positive retroperitoneal lymph nodes | |
| IIIB: Macroscopic peritoneal metastasis beyond the pelvis up to | T3b-N0/N1-M0 |
| 2 cm in greatest dimension, with or without metastasis to the | |
| retroperitoneal lymph nodes | |
| IIIC: Macroscopic peritoneal metastasis beyond the pelvis more | T3c-N0/N1-M0 |
| than 2 cm in greatest dimension, with or without metastasis to the | |
| retroperitoneal lymph nodes (includes extension of tumor to | |
| capsule of liver and spleen without parenchymal involvement of | |
| either organ) | |
| Stage IV: Distant metastasis excluding peritoneal metastases | Any T, any N, M1 |
| Stage IVA: Pleural effusion with positive cytology | |
| Stage IVB: Parenchymal metastases and metastases to extra- | |
| abdominal organs (including inguinal lymph nodes and lymph | |
| nodes outside of the abdominal cavity) | |

In some embodiments, the ovarian cancer is relapsed ovarian cancer.

In some embodiments, the ovarian cancer is high-grade serous ovarian cancer.

In some embodiments, the method of diagnosis or assessment of prognosis is performed on a subject who has previously been treated for ovarian cancer. Such treatments include one or more of: chemotherapy, surgical resection or de-bulking, radiation therapy, hormone therapy or immunotherapy.

In some embodiments of the method of diagnosis or assessment of prognosis, the ovarian cancer is relapsed ovarian cancer and the elevated level of Glypican-1 is compared to cancer tissue prior to relapse.

In some embodiments of the method of diagnosis or assessment of prognosis, the elevated level of Glypican-1 is compared to non-cancerous ovarian tissue, or comparison tissue not suspected of being cancerous. Such non-cancerous tissue can be collected from the same individual, or other individuals. In some embodiments, the comparison tissue is from the same sample as the suspected or confirmed cancer cells. In some embodiments, they are from a different ovary in the same individual.

In some embodiments, of the method of diagnosis or assessment of prognosis, the protein expression is determined by a binding agent that preferentially, or selectively, binds to Glypican 1. Such binding agents include antibodies or binding fragment of antibodies, or other such binding agents, as described herein as binding agents which could be used for the treatment of cancer. Also included are fusion proteins such as a single chain variable fragment which comprises the sequence of the variable light chain and variable heavy chain of an antibody.

### Methods of Analysing RNA

### RNA Isolation

Various methods for RNA isolation are well known in the art and the appropriate method will be selected by one skilled in the art in view of their specific requirements and constraints.

There are at least three major techniques which are extensively used in the art for RNA extraction. These are: organic extraction, such as phenol-Guanidine Isothiocyanate (GITC)-based solutions, silica-membrane based spin column technology, and paramagnetic particle technology.

A variety of commercially available kits are known in the art for RNA isolation such as AxyPrep Multisource Total RNA Miniprep (Axygen), RNeasy® Mini (Qiagen), EasySpin (Citomed), Ilustra RNAspin Mini RNA Isolation Kit (GE), TRIzol® and TRIzol plus RNA Purification System (Invitrogen) and E.Z.N.A.™ Total RNA Kit II (omega bio-tek). A comparison of the advantages, disadvantages and performance of each of these kits can be found in Tavares, L., et al. (2011), Comparison of different methods for DNA-free RNA isolation from SK-N-MC neuroblastoma, BMC Res Notes; 4, 3. Alternatively, protocols for RNA isolation are provided in Liu and Harada (2013), RNA Isolation from Mammalian Samples, Current Protocols in Molecular Biology; 103:4.16.1-4. 16.16

### Reverse Transcriptase Polymerase Chain Reaction (RT-PCR)

RT-PCR is one of the most sensitive techniques for quantifying specific nucleic acid samples.

To perform RT-PCR, RNA is extracted and purified from a tissue sample. This RNA is then reverse transcribed by a retroviral reverse transcriptase and converted to complementary DNA (cDNA). The cDNA is then combined in a buffer with a thermal stable DNA polymerase, deoxynucleotides and a forward and reverse primer and then thermally cycled to allow denaturing (separation) of double stranded DNA, annealing of primers to the separated DNA strands and extension of new DNA copies via the DNA polymerase. This process is repeated to amplify the strand of sequence between the forward and reverse primer providing short DNA sequences known as amplicons. The amplicons can then be visualised and/or quantified. Example protocols for performing RT PCR are provided in Mitchel, J. (2002) RT-PCR Protocols. Methods in Molecular Biology, Vol. 193.

### In Situ Hybridization

In situ hybridisation allows for identification and localisation of nucleic acids (such as RNA) within a biological sample. As such-unlike some other techniques—in situ hybridisation can indicate tissue distribution of nucleic acids within a sample, rather than just identifying the presence of, or quantifying the expression of, nucleic acids.

In situ hybridization utilises hybridisation between target nucleic acids (such as mRNA) with an oligonucleotide (e.g., cDNA) or RNA probe (riboprobe). Each probe is coupled with a detection moiety such as a radiolabel, enzyme, or fluorophore. Hybridisation between the complementary probe nucleic acid sequence and the target sequence can then be detected or visualised to identify the locations and amount of the target nucleotide.

Techniques for performing in situ hybridisation are known in the art. For example: Henley S. R. et al. (2021), RNA in situ hybridization for human papillomavirus testing in oropharyngeal squamous cell carcinoma on a routine clinical diagnostic platform. Journal of Oral Pathology & Medicine; 50, 1, p. 68-75.

### Nuclease Protection Assays

Techniques for performing ribonuclease protection assays are known in the art, including: Henttu P. (2001), Quantification of mRNA levels using ribonuclease protection assay. Methods in Molecular Biology; 169, p. 65-79.

### Northern Analysis

RNA samples are purified from tissue or cell samples before being separated by size via gel (e.g., agarose gel) electrophoresis under denaturing conditions (such as in the presence of formaldehyde or glyoxal/DMSO). The size separated RNA is then transferred to a membrane (such as a nitrocellulous or nylon membrane). This transfer may be done via techniques such as; capillary transfer, vacuum transfer, salt gradient or electrophoretic transfer. The RNA is then cross-linked or fixed to the membrane before being hybridized with a specific labelled probe.

Northerner blotting allows for analysis and quantification based on the transcript size. This permits analysis of different expressed variants of a gene.

Examples of northern blot techniques are provided in Brown, T et al. (2004), Analysis of RNA by Northern and Slot Blot Hybridization, Current Protocols in Molecular Biology; 4.9.1-4.9.19.

### RNA Microarray

Microarrays utilise a series of specific oligonucleotide probes immobilised in an array to a solid support. The probes at each specific location have a known sequence which will specifically hybridise to a complementary nucleic acid.

A nucleic acid sample for microarray analysis is typically prepared by reverse-transcribing isolated mRNA from a sample to create cDNA. During the reverse transcription, a fluorescent label can be added to the generated cDNA or may be added upon completion.

The labelled cDNA, from the sample to be analysed, is then incubated with the immobilised probes on the microarray under high-stringency conditions, before unhybridized cDNA is removed. The fluorescence at each location is then quantified and indicates the amount of hybridised sample nucleic acid which is complementary to each immobilised probe.

A range of commercially available microarray chips are known in the art including those manufacture by Affymetrix, Illumina, Agilent, Applied Microarrays, Eppendorf and Arrayit. Further, microarray protocols are known in the art including those provided by the National Human Genome Research Institute (https://research.nhgri.nih.gov/microarray/protocols.shtml), and Grant, G. R., et al. (2007), Analysis and Management of Microarray Gene Expression Data. Current Protocols in Molecular Biology, 77:19.6.1-19.6.30. https://doi.org/10.1002/0471142727.mb1906s77.

### RNA Sequencing (RNA-Seq)

RNA-Seq utilises next-generation sequencing platforms to analyse the sequence and expression of RNAs within cells at any given time. RNA-Seq can be used to analyses total RNA, micro RNA, transfer RNA and mRNA. Messenger RNAs are reverse-transcribed into cDNA before adapters are ligated to each end of the cDNAs. Sequencing can be done either unidirectional (single-end sequencing) or bidirectional (paired-end sequencing) with the sequences aligned in silico to a reference genome database or assembled to obtain de novo transcripts. Quantification of RNA is performed by counting the number of reads that map to each locus of the reference genome. A range of tools can be used to quantify counts including HTSeq, FeatureCounts, Rcount, Maxcounts, FIXSEQ, Cuffquant, Sailfist and Kallisto.

Differential expression between two tissues (such as cancer and non-cacner) can be calculated by known tools including DESeq, edgeR and Voom+ limma.

Protocols for performing RNA-Seq and analysing data are known in the art, including: Kukurba K. R. and Montgomery S. B. (2015), RNA Sequencing and Analysis. Cold Spring Harbor Protocols, 11:951-969; and Costa-Silva J, et al. (2017), RNA-Seq differential expression analysis: An extended review and a software tool. PLOS ONE 12 (12): e0190152. https://doi.org/10.1371/journal.pone.0190152.

### Method of Analysing Protein

### Immunohistochemistry/Immunostaining

One of the most common techniques for protein quantification and localisation is immunohistochemistry. This technique comprises fixing and mounting tissue, which is subsequently sectioned prior to incubation with primary antibodies against the protein of interest. These primary antibodies are either directly labelled or can be detected by labelled secondary antibodies. Common labels include enzymes (such a horseradish peroxidase), fluorescent-tags, radio labels or conjugates such as biotin. The label can then be detected and used to identify the location and/or quantity of the protein of interest.

Methods for performing IHC are known in the art, and include: Schlederer M, et al. (2014) Reliable Quantification of Protein Expression and Cellular Localization in Histological Sections. PLOS ONE 9 (7); Goldstein, M. and Watkins, S. (2008), Immunohistochemistry. Current Protocols in Molecular Biology, 81:14.6.1-14.6.23; and Goldstein, M. and Watkins, S. (2008), Immunohistochemistry. Current Protocols in Molecular Biology, 81:14.6.1-14.6.23.

Alternative methods for protein quantification include: High-Performance Liquid Chromatography (HPLC), Liquid Chromatography-mass spectrometry (LC/MS), Enzyme-Linked Immunosorbent Assay (ELISA), protein immunoprecipitation, immunoelectrophoresis, SDS-page and western blot. Protocols are known in the art for performing these techniques including: Mitulović G. and Mechtler K (2006), HPLC techniques for proteomics analysis—a short overview of latest developments, Briefings in Functional Genomics, 5, 4, p. 249-260; Gao Z, et al. (2009) Identification and Verification of the Main Differentially Expressed Proteins in Gastric Cancer via iTRAQ Combined with Liquid Chromatography-Mass Spectrometry. Analalytical Cellular Pathology (Amsterdam), 2019:5310684; Lorne F et al. (2001), Whole cell ELISA for detection of tumor antigen expression in tumor samples, Journal of Immunological Methods, 258, 1-2, p. 47-53; Kim, S. M., et al. (2017). Two different protein expression profiles of oral squamous cell carcinoma analyzed by immunoprecipitation high-performance liquid chromatography. World journal of surgical oncology, 15 (1), 151; Osborne C, Brooks S A. (2006) SDS-PAGE and Western blotting to detect proteins and glycoproteins of interest in breast cancer research. Methods in Molecular Medicine, 120: p. 217-29; Ni, D., Xu, P., and Gallagher, S. 2016. Immunoblotting and immunodetection. Curr. Protoc. Mol. Biol. 114:10.8.1-10.8.37; and Adams, L. D. and Gallagher, S. R. (2004), Two-Dimensional Gel Electrophoresis. Current Protocols in Molecular Biology, 67:10.4.1-10.4.23. doi: 10.1002/0471142727.mb1004s67.

Another method for quantifying protein expression on, and in, cells is flow cytometry. Briefly, a sample of tissue is taken and dissected into tissue of interest before the tissue is dissociated, digested and filtered into a single cells suspension. The cell suspension is then stained with antibodies directed against the protein of interest, such a Glypican-1 (i.e., using fluorophore-labelled primary antibodies, or a two-step labelling comprising primary antibodies followed by fluorophore-labelled secondary antibodies directed against the primary antibodies). To analyse intracellular staining, cells can be permeabilised (often after fixation) prior to staining. Cells are then processed in a flow cytometer that allows identification of cells expressing the protein of interest (such as Glypican-1) as well as quantifying the expression of the protein on each cell.

Methods for performing flow cytometry are known in the art including, El-Hajjar, L. et al., (2023) Guide to Flow Cytometry: Components, Basic Principles, Experimental Design, and Cancer Research Applications. Curr Protoc; 3 (3): e721; and Nolan, J. P. and Condello, D. (2013), Spectral Flow Cytometry. Current Protocols in Cytometry, 63:1.27.1-1.27.13.

Techniques are also known for simultaneous quantification of mRNA and protein expression, including REAP-seq and CITE-seq. Techniques known in the art include: Peterson V. et al. (2017) Multiplexed quantification of proteins and transcripts in single cells. Nature Biotechnology, 35, 936-939 https://doi.org/10.1038/nbt.3973; and Stoeckius M, et al. (2017), Simultaneous epitope and transcriptome measurement in single cells. Nature Methods, 14 (9): p. 865-868. doi: 10.1038/nmeth.4380. Epub 2017 Jul. 31. PMID: 28759029; PMCID: PMC5669064.

### Analysis Systems

The invention also provides a method of diagnosing or assessing the prognosis of a subject having ovarian cancer, the method comprising:
obtaining a sample of a suspected or confirmed ovarian cancer cells from the subject;
quantifying the expression of Glypican-1 in the sample, and alternatively in a control sample;
comparing the quantified expression of the sample from the subject to the control sample or a control value (as defined herein); and
performing analysis the compare expression of Glypican-1 in the sample compared to the control sample or the control value, wherein an elevated expression in the sample from the subject indicates ovarian cancer or a worse prognosis for the subject.

In some embodiments of the above method, the control value is stored in a computer database or on a computer system.

The methods of the invention can be performed in any suitable manner known in the art. However, in some embodiments, the comparison of the expression of Glypican-1 in a sample from a subject is compared to a control value (as defined herein) by a computer system. Preferably, the predetermined level of the control value is stored on a database. This allows the database to act as a reference for comparison against multiple samples of cancer or suspected cancer.

In such embodiments, after the level of expression of Glypican-1 is quantified, the data is input (e.g., uploaded or entered) into a computer system which compares the level of expression in the sample to the control value stored in a database. The computer system and an associated computer readable medium, can then perform any required statistical analysis which can provide a diagnosis of likelihood the sample is cancer, and/or provide an indication of the prognosis of the subject.

Accordingly, in some embodiments of the invention, there is provided a computer system that comprises a computer processor and a computer-readable medium encoded with programming instructions executable by the computer processor to compare the quantified expression of one or more of the markers and perform comparison to a control standard. Preferably, the control standard is stored within a database.

In some aspects, the present invention comprises a detection system comprising: a sample receiving section configured to receive an RNA sample from a ovarian cancer sample or a suspected ovarian cancer sample, and a detection section comprising one or more nucleic acids configured to hybridize with a Glypican-1 nucleic acid.

In some embodiments, the detection system can also include a computer system as described herein. In such embodiments, a computer-readable medium encoded with programming instructions or the computer-readable medium are executed by the computer processor to process the data associated with the detection section and determine the expression of Glypican-1 in the received sample. Further, the programming instructions may include a control value for Glypican-1 or may process data associated with a control sample to determine a control value. The programming instruction on processing the data to compare the expression of Glypican-1 in the received sample from the subject with the control value, thereby allowing the assessment of the sample to determine or predict the likelihood of the sample being ovarian cancer or assess the prognosis of subject, wherein an elevated level of Glypican-1 indicates the presence of ovarian cancer and/or indicates a worse prognosis for the subject.

### Treatment

If the patient is determined to have a cancer, such as ovarian cancer, or is determined to likely have cancer by anyone of the methods of diagnosis or determining a prognosis as described herein, then the appropriate treatment can be administered. What constitutes the appropriate treatment will be determined by a person skilled in the art and by the available and approved treatments. Currently available treatments include, but are not limited to, surgical resection or de-bulking of the cancer, systemic or local chemotherapy, systemic or local immunotherapy (as described herein), radiation or CAR T cell therapy (including the CAR described herein). Accordingly, any of the methods of diagnosis or prognosis may include a method of treatment, or my form part of a method of treatment. To avoid doubt, provided herein is a method of treating a subject confirmed or suspected of having cancer by performing a method of diagnosis or prognosis described herein.

### EXAMPLES

The invention is further described and illustrated in the following examples. The examples are only for the purpose of describing particular embodiments of the invention and are not intended to be limiting with respect to the above description and the scope of the invention as claimed in this application or future applications claiming priority from this application.

### Example 1—Analysis of Glypican-1 (GPC1) Expression in Ovarian Cancer

Analysis of microarray expression data and immunohistochemistry (IHC) demonstrated that GPC1 was increased in ovarian cancer cells and associated with negative prognosis in patients—including a lower overall survival rate and lower progression-free survival.

### Materials and Methods

### Micro Array Analysis

The GENT2 database (Park S-J, et al. (2019). GENT2: an updated gene expression database for normal and tumor tissues. BMC Medical Genomics 12 (Suppl 5), 101.DOI: 10.1186/s12920-019-0514-7) was used to assess GPC1 mRNA levels in normal tissues (ovarian surface epithelium (n=66), fallopian tube (n=40) and HGSOC tissues (n=807) based on data from annotated Gene Expression Omnibus (U133Plus2). The Kaplan-Meier plotter tool was used to assess the relationship between GPC1 mRNA expression (GPC1: 202755_s_at & 202756_s_at), and progression-free survival (PFS) and overall survival (OS) in HGSOC patients (Fekete J T et al. (2020). Predictive biomarkers of platinum and taxane resistance using the transcriptomic data of 1816 ovarian cancer patients. Gynecol Oncol 156, 654-661).

### Immunohistochemistry (IHC)

IHC was performed on tissue sections from high grade serous ovarian cancer (HGSOC) (n=37), benign (n=7), normal ovary (n=14), fallopian tube (FT, n=10) and matching HGSOC tissues at diagnosis and relapse (n=4). A tissue microarray (TMA) cohort HGSOC patients (n=101) was also assessed (Ricciardelli C et al. (2017). Keratin 5 overexpression is associated with serous ovarian cancer recurrence and chemotherapy resistance. Oncotarget 8, 17819-17832). Clinical and pathological parameters of these tissues are listed in Tables 6 and 7, respectively.

TABLE 6
Clinical and pathological characteristics
of the ovarian tissue cohort.
Normal Ovary (n = 14)

| | | |
|---|---|---|
| Age at diagnosis | Median (range) | 48 (47-64) |

Serous Benign Tumour (n = 7)

| | | |
|---|---|---|
| Age at diagnosis | Median (range) | 62 (25-80) |

Fallopian tube (n = 10)

| | | |
|---|---|---|
| Age at diagnosis | Median (range) | 58 (37-67) |

HGSOC (n = 37)

| | | |
|---|---|---|
| Age at diagnosis | Median (range) | 61 (26-95) |
| Histological Grade | Moderate | 2 |
| | Poor | 27 |
| FIGO Stage | Stage I | 1 |
| | Stage II | 3 |
| | Stage III | 21 |
| | Stage IV | 4 |

TABLE 7
Clinicopathological characteristics of high
grade serous ovarian cancer TMA cohort
Primary serous carcinomas (n = 101)

| Age at Diagnosis (years) | Median (range) | 60 | (24-86) |
|---|---|---|---|
| Histological Grade | Grade 2 | 21 | |
| | Grade 3 | 80 | |
| FIGO stage | Stage II | 2 | |
| | Stage III | 93 | |
| | Stage IV | 6 | |
| GPC1 H-index | Median (range) | 73.7 | (1.0-290.0) |

| | | |
|---|---|---|
| Recurrence | No | 24 |
| | Yes | 70 |
| | Unknown | 7 |
| Cause of Death | Ovarian cancer | 66 |
| | Other cause | 10 |
| | Alive | 24 |
| | No follow-up | 1 |

The methodology was adapted as described previously (Lokman N A et al. (2013). Annexin A2 is regulated by ovarian cancer-peritoneal cell interactions and promotes metastasis. Oncotarget 4, 1199-1211). Following citric acid antigen retrieval, tissue sections were incubated with a primary antibody: Rb polyclonal GPC1 (1/75, 16700-1-AP, Proteintech™) overnight at 4° C. Then sections were incubated with a secondary antibody (biotinylated goat anti-rabbit, 1/400, Dako™, Australia) followed by 10 streptavidin HRP (1/500, Dako™, Australia) for 1 h at room temperature. Peroxidase activity was detected using diaminobenzidine and H₂O₂ (Sigma-Aldrich™). Through Human Protein Atlas online database, kidney and liver tissues were selected as positive and negative controls, respectively. High and low GPC1 immunostaining was observed in mouse kidney and mouse liver respectively (FIGS. 13A and 13B respectively).

### Immunohistochemistry Assessment

IHC slides were scanned using the Nanozoomer™ Digital Pathology system (Hamamatsu Photonics™, Japan). Levels of GPC1 staining intensity in tumour cells were assessed using Qupath™ software (Bankhead P, et al. (2017). QuPath: Open source software for digital pathology image analysis. Scientific Reports 7, 16878. DOI: 10.1038/s41598-017-17204-5). The H-index was measured using the percentage and intensity of positively stained cancer cells on a scale of (0-300), using 3 thresholds were scored: weak (1+), moderate (2+) and strong (3+).

### Cell Culture

OVCAR3, OV90 and SKOV3 cell lines were obtained from the American Type Culture Collection (ATCC, Manassas, VA). COV362, COV318, A2780 and OAW28 cell lines were purchased from the European Collection of Authenticated Cell Cultures (ECACC). OVCAR-5 cells were obtained from Dr Thomas Hamilton (Fox Chase Cancer Center, PA, USA). Cell lines were cultured in either DMEM (Thermo Fisher Scientific™), or RPMI (Thermo Fisher Scientific™) media supplemented with 10% fetal bovine serum (FBS, Scientifix Pty Ltd) and antibiotics (100 U penicillin G, 100 μg/ml streptomycin sulfate and 0.25 μg/ml amphotericin B (Sigma Aldrich™). All cells were maintained at 37° C. under 5% CO₂ environment.

Primary HGSOC cells (n=7) were derived from the ascites fluid collected from advanced-stage HGSOC patients from the Royal Adelaide Hospital and cultured as previously described (Ricciardelli C et al. (2015). Transketolase is upregulated in metastatic peritoneal implants and promotes ovarian cancer cell proliferation. Clin Exp Metastasis 32, 441-455). Table 8 consists of the clinical and pathological parameters of these patients. Primary HGSOC cells were maintained in advanced RPMI (Life Technologies), 10% FBS, 2 mM Glutamax™ (Life Technologies) and antibiotics.

TABLE 8
Summary of clinical and pathological characteristics
of primary cell cohort and tissue explant.

| | Age at | | Tumour | Tumour |
|---|---|---|---|---|
| | Diagnosis | Diagnosis | stage | grade |
| Patient 1 | 69 | Recurrent serous papillary | 3a | 3 |
| | | carcinoma of the ovary | | |
| Patient 2 | 68 | Serous carcinoma of the ovary | 4 | 3 |
| Patient 3 | 47 | Recurrent serous carcinoma of | ND | 3 |
| | | the ovary | | |
| Patient 4 | 59 | Serous ovarian carcinoma | 3c | 3 |
| Patient 5 | 74 | Serous ovarian carcinoma | 4 | 3 |
| Patient 6 | 76 | Serous ovarian carcinoma | 4 | 3 |
| Patient 7 | 73 | Recurrent serous ovarian | 4 | 3 |
| | | carcinoma | | |
| Patient 8 | 65 | Serous carcinoma of the ovary | 4 | 3 |

### Quantitative Real-Time Reverse-Transcription PCR

Ovarian cancer cells (COV362, COV318, OAW28, OV90, OVCAR3, A2780, OVCAR5) were plated at 30,000 cells per well for 24 hr. RNAs from cells were isolated and reverse-transcribed as per manufacturer's guidelines using the TaqMan Gene expression Cells to CT™ (Life Technologies) as previously described (Lokman N A et al. (2019). 4-Methylumbelliferone Inhibits Cancer Stem Cell Activation and Overcomes Chemoresistance in Ovarian Cancer. Cancers (Basel) 11, 1187. DOI: 10.3390/cancers11081187). Complementary DNA (cDNA) was stored and used for subsequent PCR analysis using the Quantstudio 12K Flex Real Time PCR System™ (Applied Biosystems). The solutions for PCR were made to 10 μL using TaqMan™ Gene expression Master Mix (2×), GPC1 primer (Hs00892476_m1) nuclease-free water and the cDNA samples. Negative controls included samples without RNA or cDNA. PCR cycling conditions were used as previously described (Lokman N A et al. (2019). 4-Methylumbelliferone Inhibits Cancer Stem Cell Activation and Overcomes Chemoresistance in Ovarian Cancer. Cancers (Basel) 11, 1187. DOI: 10.3390/cancers11081187). Cycle threshold (CT) values were normalised to β-actin (Applied Biosystems™, Life technologies) and calibrated to it using the 2^{-ΔCT} method.

### Western Blot

Ovarian cancer cell lines and primary ovarian cancer cells (n=7) were cultured to confluency, and protein extracts were collected as described previously (Lokman N A et al. (2013). Annexin A2 is regulated by ovarian cancer-peritoneal cell interactions and promotes metastasis. Oncotarget 4, 1199-1211). Twenty micrograms of each sample was then loaded onto a 4-20% TGX gel (Bio-Rad) at 50V for 30 minutes and 110V for 90 minutes. Gels were transferred onto PVDF membranes (GE Healthcare™) overnight at 4° C. at 33V. The membranes were subsequently incubated with Rb polyclonal GPC1 (1/500, 16700-1-AP, Proteintech™) for 2 h and peroxidase-conjugated anti-rabbit IgG (1/4000, Millipore™) for 1h. Chemiluminescence (ECL Hyperfilm™, GE Healthcare) was used to visualise protein expression, scanned using Chemidoc™ MP Imaging system (Bio-Rad Laboratories™, Inc) and analysed using Imagelab™. Beta actin anti-rabbit antibody (1/5000, ab8226, Abcam™) was used as a loading control.

### Statistical Analysis

For GENT2 database, Kruskal Wallis test with Dunn's multiple comparison test was used. One-way ANOVA with Tukey's multiple comparison test was used to assess the GPC1 H-index score measured by Qupath. Kaplan Meier plotter database was used to generate survival curves and determine the relationship between GPC1 mRNA and patient outcome. Kaplan-Meier analyses with log-rank tests were performed to assess GPC1 protein relationship with progression-free survival (PFS) and overall survival (OS) (SPSS software, version 28.0, SPSS Inc, USA). A paired student's T-test was used to assess statistical significance of the H-index scores of matching HGSOC patient tissues at diagnosis and relapse.

### Results

### GPC1 is Increased in High-Grade Serous Ovarian Cancer

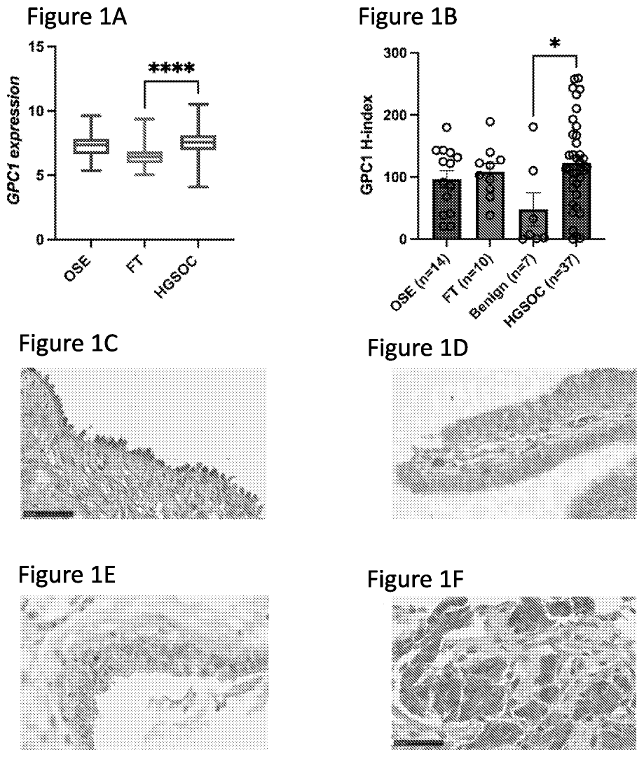

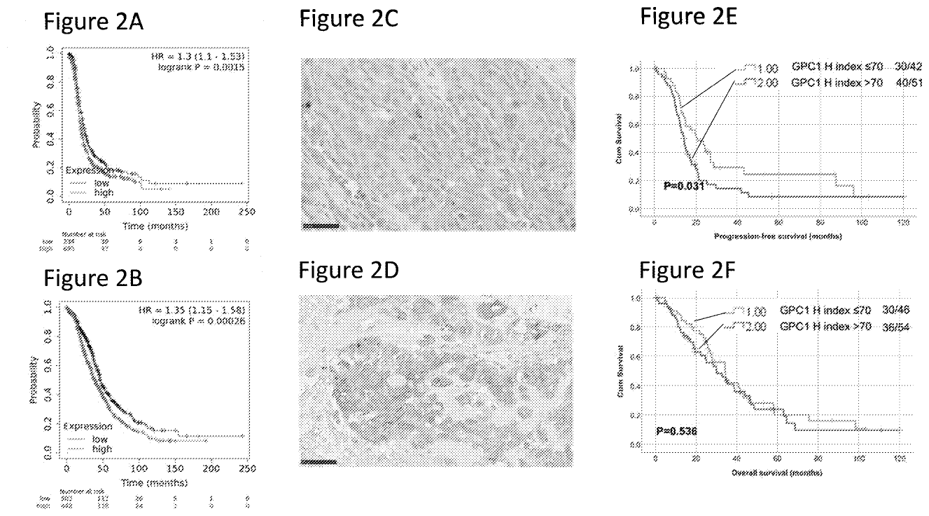

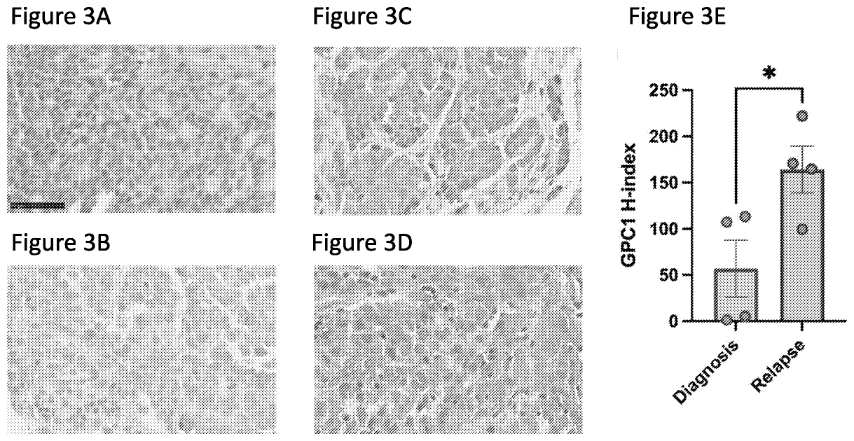

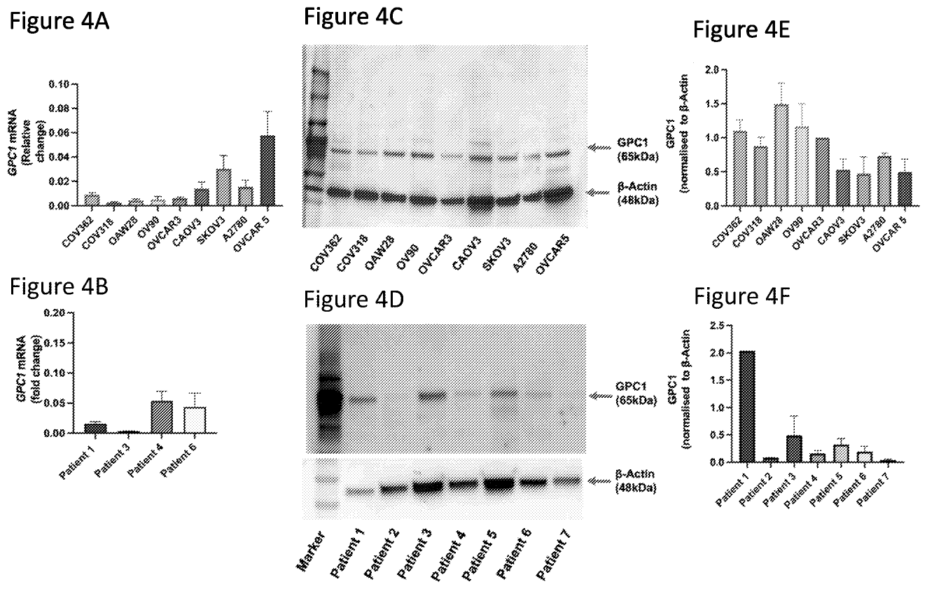

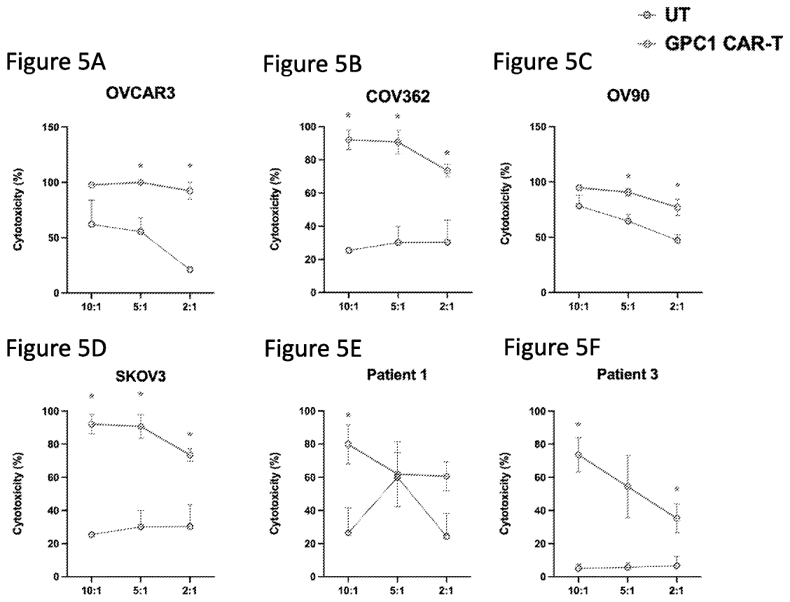

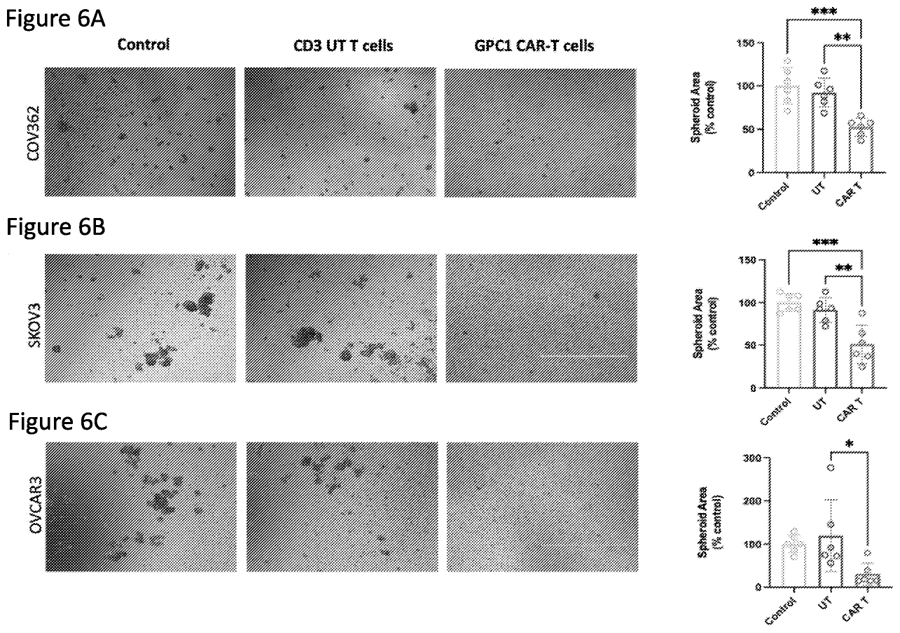

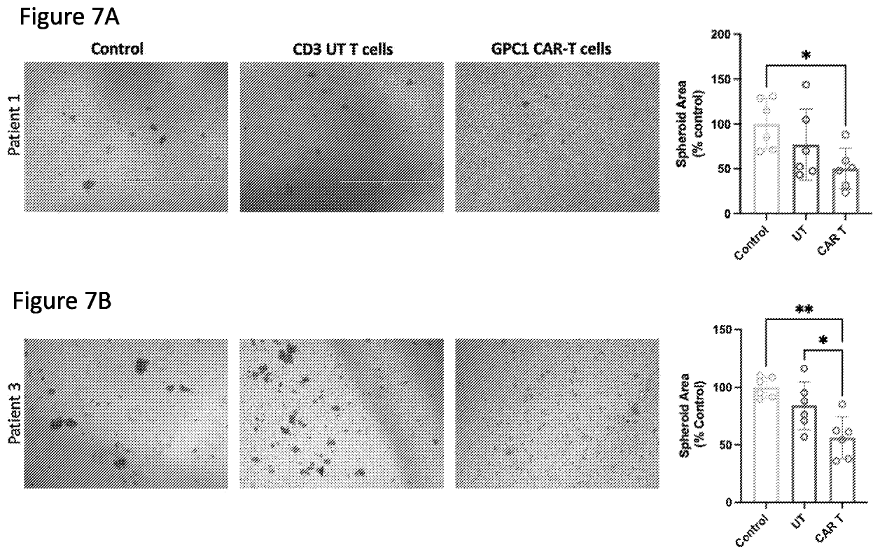

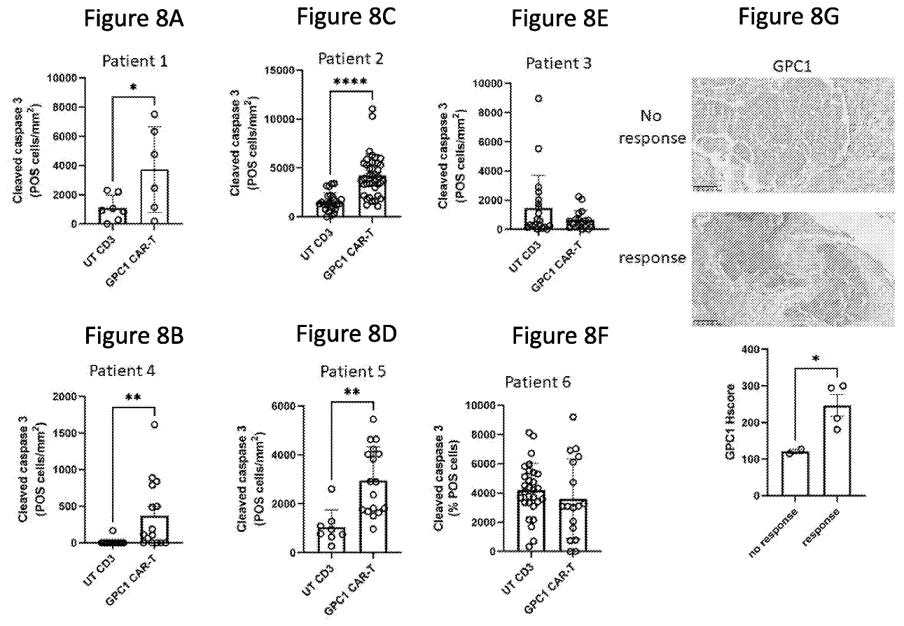

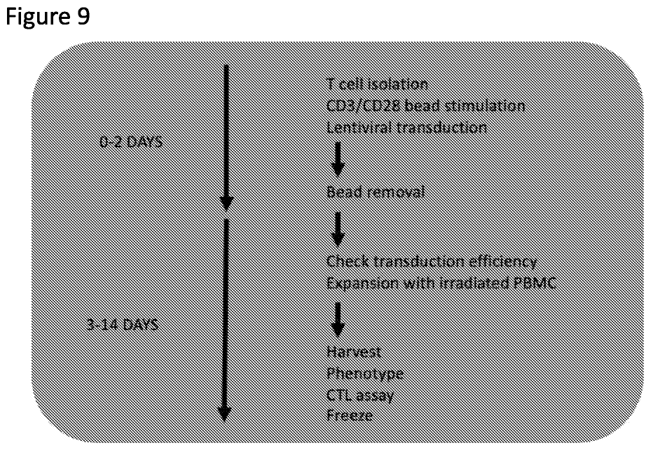

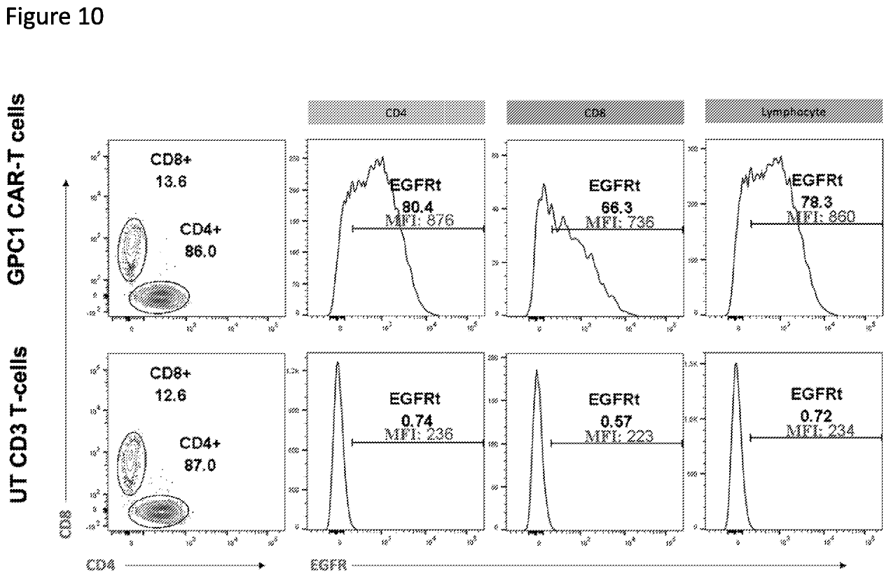

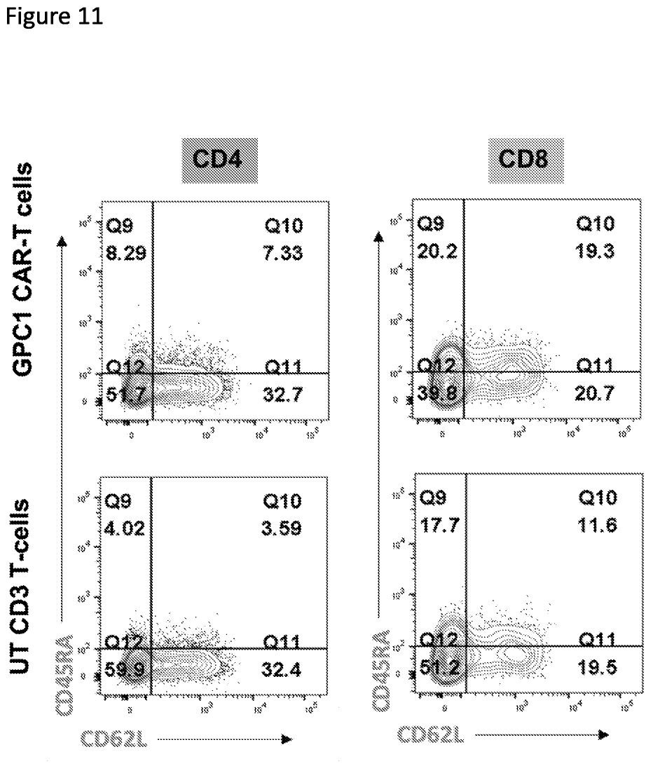

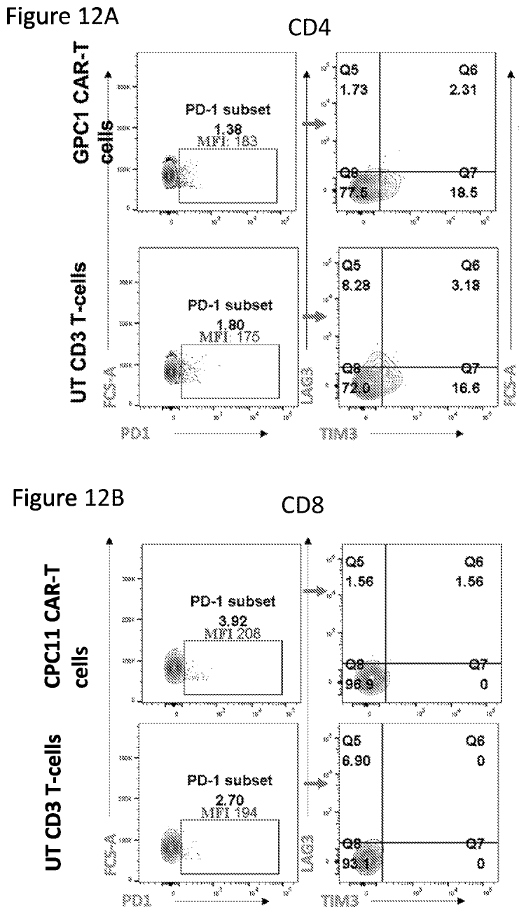

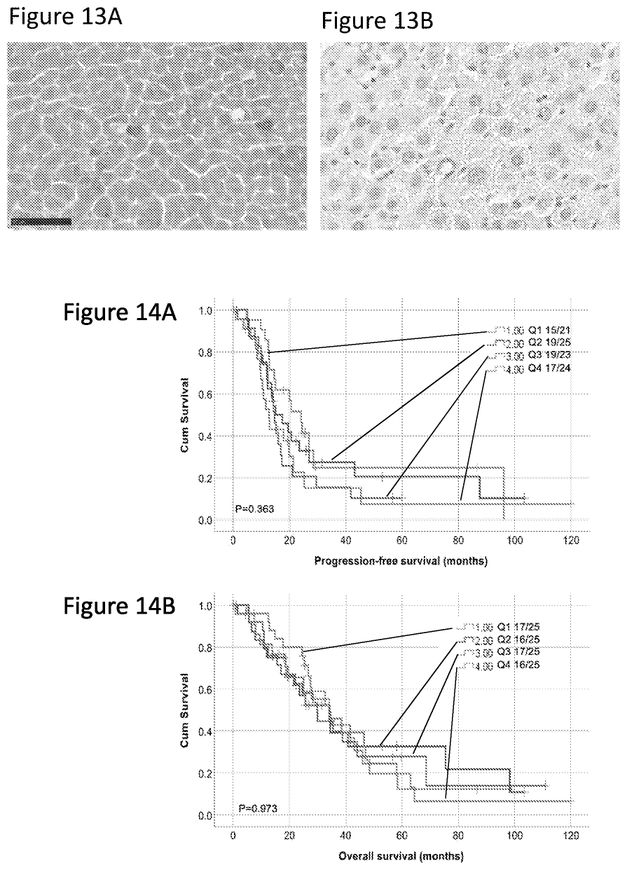

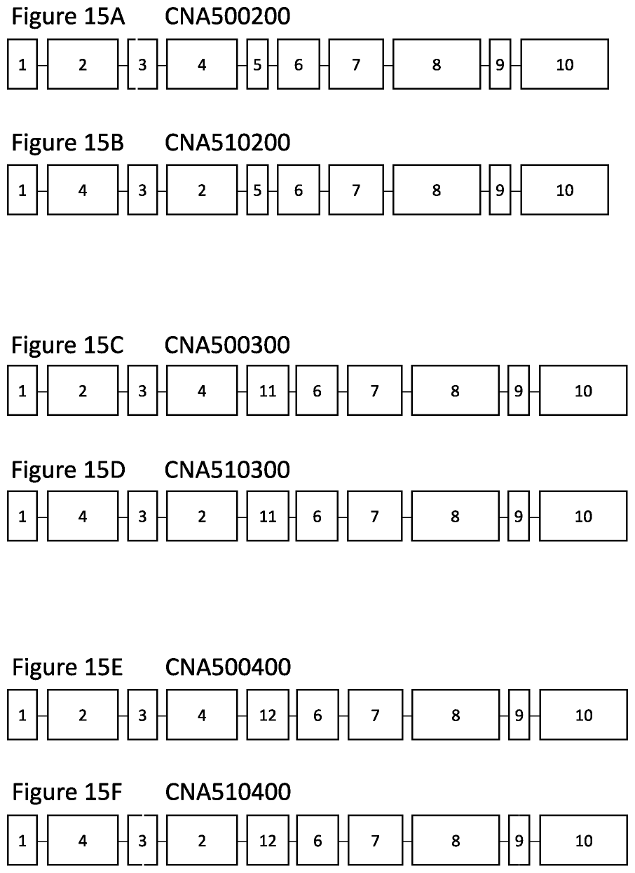

Through the analysis of the GENT2 database, GPC1 mRNA levels were significantly increased in HGSOC compared to FT (FIG. 1A, P<0.0001). However, there was no significant difference between GPC1 expression in ovarian surface epithelium (OSE) and HGSOC (FIG. 1A). Immunohistochemistry (IHC) assessment of GPC1 protein levels measured as an H-index was significantly increased in HGSOC compared to benign serous cystadenoma (FIG. 1B). However, no difference was observed with HGSOC GPC1 H-index compared with either OSE or FT. Representative images demonstrate cytoplasmic and membrane GPC1 staining in OSE (FIG. 1C), FT (FIG. 1D) and low GPC1 staining in benign serous cystadenoma tissue (FIG. 1E). High GPC1 cytoplasmic and membrane staining was present in the HGSOC tissues (FIG. 1F).

High GPC1 Expression is Associated with Poor Patient Outcome

Survival curves were generated using Kaplan Meier online plotter tool to investigate the relationship between GPC1 mRNA levels and patient outcome (FIGS. 2A to 2F). High GPC1 mRNA expression was significantly associated with reduced PFS (FIG. 2A, Hazard Ratio (HR)=1.3, p=0.0015), and OS (FIG. 2B, HR=1.35, p=0.00026).

GPC1 protein levels were assessed in a TMA cohort of HGSOC. The median H-index value observed for this cohort was 73.7. Examples of HGSOC tissues with low GPC1 protein expression and high expression are shown in FIGS. 2C and 2D, respectively. GPC1 H-index was separated into quartiles for the initial Kaplan-Meier survival analyses (FIG. 14). For the PFS analysis, there was a separation between the higher quartiles (Q3 and Q4) and the low quartiles (Q1& Q2), but no separation was observed for the OS analysis. Using the H-index score with a cut point of 70 (close to the median value), GPC1 levels>70 were associated with reduced PFS (FIG. 2E, P=0.031) but not OS (FIG. 2F, p=0.536).

### GPC1 Expression is Elevated Following Relapse

GPC1 protein levels were assessed in matching HGSOC tissues at diagnosis and at relapse. Examples of GPC1 immunostaining in HGSOC tissues at diagnosis are shown in FIGS. 3A and 3B, and a matching tissue at relapse FIGS. 3C and 3D. The quantification of the IHC staining using QuPath indicates that GPC1 levels were increased in matching tumour tissues at relapse compared to tissues at diagnosis (FIG. 3E, P=0.0014, paired T-test).

### GPC1 Expression in Ovarian Cancer Cells

Quantitative PCR (qRT-PCR) showed that GPC1 mRNA was expressed in all ovarian cancer cell lines (FIG. 4A) and primary HGSOC cells (FIG. 4B). The highest GPC1 expression was observed in OVCAR5 and patient 4 primary cells. Western blots detected a band at a predicted molecular weight of 65 kDa confirming the expression of GPC1 in the ovarian cancer cell lines (FIG. 4C) and primary HGSOC cells (FIG. 4D). The western blot quantitation showed the highest GPC1 protein levels in OAW28, and OV90 (FIG. 4E). Primary cells from patient 1 and patient 3 isolated from HGSOC patients with recurrent disease expressed the highest GPC1 protein levels (FIG. 4F). Ovarian cancer cell lines with a range of GPC1 levels (OVCAR3, OV90, COV362 and SKOV3) and primary cells (Patient 1 and Patient 3) were selected for further in vitro studies.

### Discussion of Results

The above results indicated that: i) GPC1 mRNA and protein expression was increased in HGSOC compared to non-cancer tissues although these levels were not necessarily correlated within a patient or within a cell line; ii) Increased GPC1 mRNA levels were associated with PFS and OS; and iii) High GPC1 protein expression levels in the tumour cells were associated with reduced PFS.

Investigating the expression of GPC1, it was shown that GPC1 mRNA expression was significantly increased in HGSOC compared to the FT, which is the site of origin for HGSOC. Further, GPC1 protein expression was significantly increased in HGSOC compared to benign tissues.

The results from the Kaplan Meier plotter analysis and TMA cohort demonstrated a significant relationship between high GPC1 expression and poor outcome in patients with HGSOC. We only observed faint GPC1 staining in the stroma with GPC1 predominantly localised in the cell membrane and cytosol.

The results also demonstrate that GPC1 was expressed to varying degrees in the ovarian cancer cell lines and primary cells.

### Example 2—Anti-Glypican-1 Chimeric Antigen Receptor T-Cells Effectively Kill Ovarian Cancer Cells

Having demonstrated GPC1 expression in ovarian cell cancer cells, especially HGSOC, it was determined if ovarian cancer cells could be targeted with agent that induced killing of GPC1 expressing cells. To target GPC1 expressing cells, anti-GPC1 CAR-T cells were developed and used in cancer cell lysis assays. These experiments demonstrated that ovarian cancer cells can be killed by anti-GPC1 agents, such as CAR-T cells.

### CAR-T Cell Manufacturing and Characterisation.

CNA500200 CAR-T cells were generated in Prof. Simon Barry's laboratory using established protocols (Jensen M C & Riddell S R (2015). Designing chimeric antigen receptors to effectively and safely target tumors. Curr Opin Immunol 33, 9-15; Wang X, et al. (2012). Phenotypic and functional attributes of lentivirus-modified CD19-specific human CD8+ central memory T cells manufactured at clinical scale. J Immunother 35, 689-701; and WO2022/104424A). A GPC1 binding domain has been cloned into a second-generation CAR backbone encoding a linker and intracellular domains CD3, CD28 and epidermal growth factor (EGFR) reporter (Jensen M C & Riddle S R (2015), supra and Wang X et al. (2012), supra).

Lentivirus was produced by transfecting 293T cells with third generation self-inactivating lentiviral plasmids and packaging plasmids encoding REV, VSV-G and gag-pol using established methods (Barry S C et al. (2000). Lentiviral and murine retroviral transduction of T cells for expression of human CD40 ligand. Hum Gene Ther 11, 323-332). A brief overview of the transduction protocol is illustrated in FIG. 9.

Full characterisation using Fluorescent Activated Cell Sorting (FACs) was conducted on the CAR-T cells used for the assays by Batjargal Gundsambuu (Molecular Immunology, University of Adelaide). The transduction efficiency was measured by EGFR expression. Within the CD4 T cell population, 80.4% of cells were EGFR-positive, in the CD8 T cell population 66.3% of cells were positive, and in the total lymphocyte populations 78.3% were positive (FIG. 10).

Markers for cell maturation, CD45RA and CD62L were also assessed. In the CD4 population, 51.7% of cells displayed an effector memory T-cell (T_{EM}) phenotype, 32.7% displayed a central memory T-cell (T_{CM}) phenotype, 8.29% displayed an effector memory cells re-expressing CD45RA (T_{EMRA}) phenotype and 7.3% of cells expressed a naïve T-cell phenotype (FIG. 11). While the CD8 cell population had a slightly more naïve phenotype (19.3%), 20.7% expressing a central memory T-cell phenotype, 29.8% expressing the effector memory T-cell phenotype and 20.2% expressing the T_{EMRA} phenotype (FIG. 11).

An exhaustion staining panel was also assessed using PD1, LAG3 and TIM3 antibodies. FACs analysis shows that within the CD4 population, 1.38% of cells express very low levels of PD1, of these cells, only 2.31% of cells expressed LAG3 and TIM3 (FIG. 12). In the CD8 population, 3.92% of cells express PD1, of these cells only 1.56% express LAG3 and TIM3. These results indicate low levels of T-cell exhaustion.

### Materials and Methods

### MTT Cell Survival Assay

SKOV3, COV362, OVCAR3, OV90 and primary HGSOC cells (n=2) were plated at 10,000 cells/well in 96-well plates in respective growth media. After 24 h, cells were treated with either (i) control media, (ii) untransduced (UT) CD3 T-cells or (iii) anti-GPC1 CAR T-cells (effector T cells: target cancer cell ratio (E:T) at ratios 2:1, 5:1 and 10:1) for 48 h. The cell monolayer was washed twice with RPMI media to remove T cells. MTT assay was conducted as described previously (Ricciardelli C, et al. (2013). Chemotherapy-induced hyaluronan production: a novel chemoresistance mechanism in ovarian cancer. BMC Cancer 13, 476. DOI: 10.1186/1471-2407-13-476).

### Spheroid Assays

SKOV3, COV362, OVCAR3 and primary ovarian cancer cells (n=2), were plated at 20,000 cells/well on poly-HEMA (30 mg/mL in 95% ethanol, Sigma Aldrich™) coated 24 well plates in respective growth media. After 24 hrs, cells were treated with X-VIVO 15 medium (Lonza™, 5% Human Serum-Sigma-Aldrich™, 2 mM L-Glutamine-Sigma™, 20 mM HEPES) or UT CD3 T-cells or anti-GPC1 CAR-T cells (E:T ratio of 5:1). Spheroid formation was observed over 6 days and bright field images were taken using the EVOS® light microscope FL imaging system (Life Technologies™). Spheroid area (μm²) was measured for spheroids greater than 50 μm in diameter for each of the treatment groups (n=5 images/well in duplicate wells from three independent experiments) using Image J 32 software (Image J I.50i, National Institute Health, Bethesda, MD, USA) as described previously (Lokman N A, et al. (2019). 4-Methylumbelliferone Inhibits Cancer Stem Cell Activation and Overcomes Chemoresistance in Ovarian Cancer. Cancers (Basel) 11, 1187).

### Patient-Derived Explant (PDE) Assays

Tissues were collected at surgery and cryopreserved in liquid nitrogen containing 15% DMSO and 25% FBS. The cryopreserved tissue from patients (FIG. 11) was dissected into 1 mm³ pieces, explanted onto gelatine dental sponges (Spongostan™, Johnson & Johnson™) in CD3 T cell X-VIVO media (with cytokines IL-2 (50U/mL), IL-7 (5 ng/ml), and IL-15 (0.5 ng/ml)) and treated with either (i) control media, (ii) anti-GPC1 CAR-T cells (2×10⁶/mL) or (iii) UT CD3 T-cells (2×10⁶/ml) in a humidified atmosphere at 37° C. containing 5% CO₂. Tissues were then collected and fixed with formalin after 72 h and processed for histology. Cell apoptosis was measured using cleaved caspase 3 as described previously (Ricciardelli C et al. (2018). Novel ex vivo ovarian cancer tissue explant assay for prediction of chemosensitivity and response to novel therapeutics. Cancer Lett 421, 51-58).

### Results

### Effect of GPC1 CAR-T Cells on Ovarian Cancer in Monolayer In Vitro.

All ovarian cancer cells and primary cells responded to treatment with GPC1 CAR-T cells. A significant decrease in cell survival was observed at 5:1 and 2:1, E:T ratio, compared to UT CD3 T-cells for OVCAR3 (FIG. 5A), COV362 (FIG. 5B), OV90 (FIG. 5C) and SKOV3 (FIG. 5D) cell lines. Anti-GPC1 CAR-T cells also reduced COV362 (FIG. 5B) and SKOV3 cell (FIG. 5D) survival at 10:1 compared to UT CD3 T-cells. Patient 1 primary cells displayed a statistically significant decrease in cell survival when incubated with anti-GPC1 CAR-T cells at an ET ratio of 10:1 (FIG. 5E) but not at 5:1 and 2:1. Anti-GPC1 CAR-T cells also exhibited statistically significant effects at E:T ratios of 10:1 and 2:1 on the survival of patient 3 primary cells but not at 5:1 (FIG. 5F).

### Effect of GPC1 CAR-T Cells on Ovarian Cancer in 3D Spheroid Formation

Spheroids comprising the cell lines COV362 (FIG. 6A), SKOV3 (FIG. 6B) and OVCAR3 (FIG. 6C) responded to anti-GPC1 CAR-T cell treatment at the E:T ratio of 5:1, resulting in a statistically significant decrease in the spheroid area compared to UT CD3 T-cells. Significant differences were also observed between control and anti-GPC1 CAR-T cells for spheroids comprising COV362 (FIG. 6A) and SKOV3 (FIG. 6B) cells but not OVCAR3 cells (FIG. 6C). A statistically significant reduction in spheroid size was observed between UT CD3 T-cells and anti-GPC1 CAR-T cells for spheroids consisting of cells of patient 3 (FIG. 7B) but not for spheroids from patient 1 (FIG. 7A), although a reduction was observed for this patient. The spheroid size was statistically significantly reduced with anti-GPC1 CAR-T cell treatment for both patient 1 (FIG. 7A) and patient 3 (FIG. 7B), compared to control media. None of the cells demonstrated a statistically significant difference between UT CD3 T-cell treatment and control treatment.

### Effect of GPC1 CAR-T Cells on Patient-Derived Explants

Ovarian cancer tissue from 6 patients was selected for a PDE assay (FIG. 8A to 8F). The PDE assay was performed by treating the explant patient tissue with UT CD3 T-cells or anti-GPC1 CAR-T cells, followed by a cleaved caspase-3 immunostaining to assess apoptosis in the explant tissue.

There was a statistically significant increase in cleaved caspase 3 staining in patients 1 to 4 (FIGS. 8A to 8D) indicating increased cell death in the explant tumour tissue following treatment with anti-GPC1 CAR-T cells compared to UT CD3 T-cells. Comparison of GPC1 expression in tissue that did respond to CAR T cell treatment with unresponsive tissue (e.g., patients 5 and 6) showed low levels of GPC1 expression in the explant that did not respond to CAR T cell treatment (FIG. 8G) indicating GPC1 expression was associated with treatment outcomes.

### Discussion of Results

The effects of anti-GPC1 CAR-T cells were assessed in ovarian cancer cell lines with varying GPC1 expression, as well as in two primary cells from patients with recurrent ovarian cancer.

The results from the 2D monolayer assay indicated that anti-GPC1 CAR-T cells exhibited killing effects in a dose-dependent manner.

Further significant decreases in cancer cell survival were observed at the lowest concentration of 2:1 ratio for all ovarian cancer cells except for primary cells from patient 1. Statistically significant effects at the 10:1 ratio were not observed for OVCAR3 and OV90, most likely due to increased cytotoxic effects with a higher number of UT CD3 T-cells, but the data indicated that there was still an increase in killing at these ratios.

The assessment of CAR-T cell efficacy in 3D spheroid assay allowed for accurate physiological representation of the tumour microenvironment, particularly when such structure form in malignant ascites in ovarian cancer patients.

This study showed significant anti-tumour activity of anti-GPC1 CAR-T cells in vitro against primary ovarian cancer 3D spheroids.

Anti-GPC1 CAR-T cell efficacy was also investigated in an ex-vivo model, PDE assay, which importantly maintains tissue architecture and viable tumour cells like its native tissue. The results indicated that anti-GPC1 CAR-T cells were effective at inducing apoptosis in HGSOC patient tissue, compared to UT CD3 T-cells, indicating killing of target cancer cells.

In summary these results show that i) GPC1 CAR-T cells had anti-tumour activity against ovarian cancer cell lines in a monolayer assay and a 3D spheroid assay; ii) GPC1 CAR-T cells had anti-tumour activity against primary ovarian cancer cells isolated from HGSOC patients following disease relapse in a monolayer assay and a 3D spheroid assay; and iii) GPC1 CAR-T cells were effective at inducing apoptosis in PDE assays. Together these findings indicate that GPC1 CAR-T cells may provide a novel immunotherapy against ovarian cancer, in particular HGSOC.

TABLE 9
List of Abbreviations

| | |
|---|---|
| CAR | Chimeric antigen receptor |
| cDNA | Complimentary DNA |
| CT | Cycle threshold |
| DMSO | Dimethyl sulfoxide |
| DNA | Deoxyribonucleic acid |
| E:T | Effector cell to target cell ratio |
| FACS | Fluorescent activated cell sorting |
| FCS-A | Forward scatter area |
| FBS | Fetal Bovine Serum |
| FT | Fallopian tube |
| GPC1 | Glypican-1 |
| HGSOC | High grade serous ovarian cancer |
| HR | Hazard ratio |
| IHC | Immune histochemistry |
| IL | Interleukin |
| LAG3 | Lymphocyte activation gene 3 |
| mRNA | Messenger RNA |
| OS | Overall survival |
| OSE | Ovarian surface epithelium |
| PCR | Polymerase chain reaction |
| PD1 | Programme cell death protein 1 |
| PDE assay | Patient-derived explant assays |
| PFS | Progression-free survival |
| RNA | Ribonucleic acid |
| T_{CM} | Central memory T-cell |
| T_{EM} | Effector memory T-cell |
| T_{EMRA} | Effector memory cells re-expressing CD45RA |
| TIGIT | T cell immunoreceptor with Ig and ITIM domains |
| TIM3 | T cell immunoglobulin and mucin-domain containing-3 |
| TMA | Tissue microarray |
| UT | Untransduced |

### Definitions and Qualifications

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be further understood that terminology such as “comprise”, or variations such as “comprises” or “comprising” inherently include within their scope (without being limited to) versions of the invention that excludes other elements directly related to the invention. Accordingly, terminology such as “consisting of” or “consisting essentially of” can be substituted for terminology such as “comprise”, “comprises” or “comprising” with the effect of limiting the scope of the invention to the specifically recited elements. Notably, where it is explicitly intended for the invention to be considered in an exhaustive manner, such limitations should be considered to relate only to the inventive concept disclosed herein and other features can be added which fall outside of the scope of the inventive concept. Such features or elements may include, but are not limited to, excipients, formulations, additives, diluents, packaging, adjuvants, and collocated features which are not to be excluded by terminology such as “consisting of” or “consisting essentially of”.

Referenced documents, publications and patents are to be included in their entirety by way of reference. The teachings and disclosures in such documents, publications and patents are therefore considered to form part of the disclosure of this specification.

All methods described herein can be performed in any suitable order unless indicated otherwise herein or clearly contradicted by context. The use of any and all examples, or exemplary language (e.g., “such as”, “i.e.”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Future patent applications may be filed on the basis of claiming priority from the present application, or being continuations of, or having divisional status from, the present application. It is to be understood that the following claims are not intended to limit the scope of what may be claimed in any such future application. Features may be added to or omitted from the claims at a later date so as to further define or re-define the invention or inventions claimed.

It will be apparent to the person skilled in the art that while the invention is described herein in detail for the purposes of clarity and understanding, various modifications and alterations to the embodiments and methods described herein may be made without departing from the scope of the inventive concept disclosed in this specification.

1. A Chimeric Antigen Receptor (CAR) comprising an antigen-recognition domain, a transmembrane domain, and a signalling domain, wherein the antigen-recognition domain recognises Glypican-1.

2. The CAR of claim 1, wherein the antigen-recognition domain comprises a binding-portion of an antibody that recognises Glypican-1.

3. The CAR of claim 1 or claim 2, wherein the portion of the antibody that recognises Glypican-1 is selected from the group consisting of a fragment-antigen binding (Fab), a variable heavy chain of an antibody or a variable light chain of an antibody.

4. The CAR of any one of claims 1 to 3, wherein the antigen-recognition domain is a single chain variable fraction (scFv) which has sequence identity to an antibody that binds to Glypican-1.

5. The CAR of any one of claims 1 to 4, further comprising a linker between the antigen-recognition domain and the transmembrane domain.

6. A cell comprising the CAR of any one of claims 1 to 5.

7. A cell according to claim 6, wherein the cell is an immune cell.

8. The cell of claim 6 or claim 7, wherein the cell is a lymphocyte.

9. The cell of any one of claims 6 to 8, wherein the cell is a CD3+ cell.

10. The cell of any one of claims 6 to 9, wherein the cell is a CD8+ cell or a CD4+ cell.

11. The cell according to any one of claims 6 to 8, wherein the cell is a natural killer (NK) cell or a natural killer T (NKT) cell.

12. A method of diagnosing or assessing the prognosis of a subject having ovarian cancer, the method comprising determining the level of Glypican-1 in ovarian cells from the subject, wherein an elevated level of Glypican-1 indicates the presence of ovarian cancer and/or indicates a worse prognosis for the subject.

13. The method of claim 12, wherein the ovarian cancer is relapsed ovarian cancer.

14. The method of claim 12 or claim 13, wherein the ovarian cancer is high-grade serous ovarian cancer.

15. The method of any one of claims 12 to 14, wherein the subject has previously been treated for ovarian cancer with one or more of: chemotherapy, surgical resection or de-bulking, or radiotherapy.

16. The method of any one of claims 12 to 15, wherein the ovarian cancer is relapsed ovarian cancer and the elevated level of Glypican-1 is compared to cancer tissue prior to relapse.

17. The method of any one of claims 12 to 16, wherein the elevated level of Glypican-1 is compared to non-cancerous ovarian tissue.

18. The method of any one of claims 12 to 17, wherein determining the level of Glypican-1 comprises quantifying Glypican-1 protein expression and/or mRNA expression.

19. The method of claim 18, wherein quantifying Glypican-1 protein expression comprises quantifying the surface expression of the Glypican-1 protein.

20. The method of claim 18, wherein determining the level of Glypican-1 protein expression comprises quantifying the intracellular expression of the Glypican-1 protein.

21. The method of any one of claims 18 to 20, wherein the protein expression is determined by an agent that preferentially, or selectively, binds to Glypican 1.

22. The method of claim 21, wherein the agent that binds to Glypican-1 is an antibody or a binding fragment thereof.

23. The method of claim 21, wherein the agent that binds to Glypican 1 is a single chain variable fragment comprising the sequence of the variable light chain and variable heavy chain of an antibody.

24. The method of claim 22 or claim 23, wherein the antibody is MIL-38.

25. A method of treating a subject having ovarian cancer, the method comprising killing cells expressing Glypican-1.

26. The method of claim 25, wherein the cells expressing Glypican-1 are determined to express an elevated level of Glypican-1 protein.

27. The method of claim 26, wherein the elevated level of Glypican-1 protein expression includes elevated surface expression of the Glypican-1 protein.

28. The method of claim 26, wherein the elevated level of Glypican-1 protein expression includes elevated intracellular expression of Glypican-1 protein.

29. The method of any one of claims 25 to 28, wherein the ovarian cancer is relapsed ovarian cancer.

30. The method of any one of claims 25 to 29, wherein the ovarian cancer is high-grade serous ovarian cancer (HSOC).

31. The method of any one of claims 25 to 30, wherein the subject has previously been treated for ovarian cancer with one or more of chemotherapy, surgical resection or de-bulking, or radiotherapy.

32. The method of any one of claims 26 to 31, wherein the ovarian cancer is relapsed ovarian cancer and the elevated level of Glypican-1 is compared to cancer tissue prior to relapse.

33. The method of any one of claims 26 to 31, wherein the elevated level of Glypican-1 is compared to non-cancerous ovarian tissue.

34. The method of any one of claims 25 to 33, wherein the cells are killed by administering to the subject an agent that preferentially, or selectively, binds to Glypican 1.

35. The method of claim 34, wherein the agent that binds to Glypican-1 is an antibody or a binding fragment thereof.

36. The method of claim 34, wherein the agent that binds to Glypican 1 is a single chain variable fragment comprising the sequence of the variable light chain and variable heavy chain of an antibody.

37. The method of claim 35 or claim 36, wherein the antibody is MIL-38.

38. The method of claim 34, wherein the agent that binds to Glypican-1 is a chimeric antigen receptor (CAR) expressing cell.

39. The method of claim 38, wherein the CAR expressing cell is a cell according to any one of claims 6 to 11.

40. The method of any one of claims 25 to 39, comprising performing the method of claims 12 to 24, prior to killing the cells expressing Glypican-1.
